(19) United States
(12) Patent Application Publication
(10) Pub. No.: US 2026/0347847 A1
(43) Pub. Date: Nov. 5, 2026

(54) DOSING REGIMEN FOR HUNTINGTON TREATMENT

(71) Applicant: VICO Therapeutics B.V., Leiden (NL)

(72) Inventors: Nicole Anne DATSON, Leiden (NL); Scott Allan SCHOBEL, Leiden (NL)

(21) Appl. No.: 19/375,474

(22) Filed: Oct. 31, 2025

Related U.S. Application Data
(63) Continuation of application No. PCT/EP2025/076012, filed on Sep. 12, 2025.

(30) Foreign Application Priority Data
Sep. 12, 2024 (EP) 24199964.8
Apr. 30, 2025 (EP) 25173650.0

(51) Int. Cl. C12N 15/113; A61K 9/00; A61P 25/28

(52) U.S. Cl. CPC C12N 15/113; A61K 9/0019; A61P 25/28; C12N 2310/315; C12N 2310/321; C12N 2310/3341

(57) ABSTRACT

The invention relates to the field of human genetics, more specifically neurological disorders. The invention in particular relates to the use of the antisense oligonucleotide AON1 with improved characteristics enhancing clinical applicability for treating Huntington as further defined herein.

### RELATED APPLICATIONS

This application is a continuation of International Patent Application No. PCT/EP2025/076012, filed Sep. 12, 2025, which claims priority to European Patent Application Nos. 25173650.0, filed Apr. 30, 2025, and 24199964.8, filed Sep. 12, 2024, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Oct. 31, 2025, is named 771334_VCO9-005PCCON_ST26.xml and is 86,326 bytes in size.

### FIELD

The invention relates to the field of human genetics, more specifically neurological disorders. The invention in particular relates to the use of the antisense oligonucleotide (AON) (CUG)7 consisting of 2′-O-methyl phosphorothioate RNA wherein all of its cytosines have been replaced by 5-methylcytosine, also named AON1 (i.e. SEQ ID NO:1) for treating Huntington disease (HD) as further defined herein.

### BACKGROUND OF THE INVENTION

Neurological disorders or neuropathies are characterized by neurodegeneration and impaired nerve control leading to problems with movement, spasticity or paralysis. Examples include HD, several types of spinocerebellar ataxia (SCA), Friedreich's ataxia (FA), Amyotrophic Lateral Sclerosis (ALS) and Frontotemporal dementia (FTD). A subset of neuropathies is caused by a cis-element repeat instability.

HD has the prevalence of approximately 5.7 per 100,000 in Europe and North America (Pringsheim T; et al (2012), Mov. Disord., 27(9): p. 1083-1091). It is caused by a CAG repeat expansion in the first exon of the HTT gene located on chromosome four resulting in a polyglutamine expansion in the encoded huntingtin protein (HTT). Expansion of these repeats results in expansion of a glutamine stretch at the N-terminal end of the 348 kDa cytoplasmic huntingtin protein. Huntingtin has a characteristic sequence of 26 or fewer glutamine amino acid residues in the normal form; the mutated huntingtin causing the disease has 36 or more residues. Individuals with CAG repeats in the range from 27 to 35 CAG repeats are not at risk of developing symptoms of HD but, because of instability in the CAG tract, may be at risk of having a child with an allele in the HD-causing range (Semaka A. et al., 2006, Clin Genet., 70:283-94). CAG repeats in the range of 36 to 39 have reduced-penetrance and may not develop symptoms. Full-penetrance HD-causing alleles contain 40 or more CAG repeats. Many pathogenic mechanisms have been hypothesised for the apparent toxic gain-of-function of this polyglutamine-expanded protein, including abnormalities in cellular proteostasis, altered gene transcription, mitochondrial dysfunction and oxidative stress, excitotoxicity, synaptic and neuronal failure, deficient axonal transport, spread of mutant HTT from cell-to-cell in a prion-like fashion and loss of trophic support (Kuemmerle S, et al., Ann Neurol, 1999. 46(6): p. 842-849, Moumné L, et al, Front Neurol, 2013. 4: p. 127, and Ross C A, et al., Nat Rev Neurol, 2014. 10(4): p. 204-216). Mutant HTT mRNA transcripts have also been shown to contribute to neuronal toxicity (Bañez-Coronel M, et al., PLoS Genet, 2012. 8(2): p. e1002481). CAG repeat expansion has been shown to result in aberrantly spliced exon 1 mRNA fragments that are translated into a short exon 1 HTT protein that is toxic to neurons (Sathasivam K. et al., 2013, Proc Natl Acad Sci USA, 110(6):2366-70). Repeat-associated non-ATG (RAN) translation of the CAG repeat in both the sense and antisense direction resulting in the formation of toxic homoploymeric proteins polyGln, polyAla, polySer, polyCys and polyLeu has also been suggested to play a role in the pathogenesis of HD (Bañez-Coronel M. et al., 2015, Neuron, 88(4):667-77). Somatic CAG repeat expansion resulting in ultra long (>100->1000) CAG repeats has been shown to occur in the brain of HD patients and is associated with drives the rate of pathogenesis (Kennedy L. et al. 2003, Human Molecular Genetics, 12(24):3359-3367).

The continuous expression of mutant huntingtin molecules in neuronal cells results in the formation of large protein deposits which eventually give rise to cell death, especially in the frontal lobes and the basal ganglia (mainly in the caudate nucleus). The severity of the disease is generally proportional to the number of extra residues.

Despite the seriously debilitating nature of HD, there is currently no approved disease-modifying treatments for HD or any other polyQ disease.

For HD various symptomatic treatments are available, but their use is often limited by a plethora of adverse effects, and importantly, none are proven to alter the course of the disease. Thus, there is a high unmet medical need for effective disease-modifying therapies for patients with devastating progressive disorder as HD.

Since the polyQ protein implicated in HD as well as in the other polyQ diseases act through a dominant gain-of-function mechanism resulting in neurotoxicity, suppression of the respective mutant protein using antisense oligonucleotides targeting CAG repeat expansions is an appealing and promising approach to slow or halt disease progression in sufferers from HD.

Preclinical data using AON1 (as described in WO 2013/162363) seem to suggest that this type of molecule can be applied to effectively reduce reduce toxic Huntintin protein levels in HD patient-derived cells.

However, so far no evidence has been provided that such molecule could effectively reduce mutant Huntingtin HTT levels in cerebrospinal fluid of Huntington patients. The present trial is the first-in-human (FiH) evaluation of AON1 demonstrating its significant effect on the mutated HTT protein and on a marker specific for neurodegeneration only 28 days following a single dose of 40 mg of AON1 in a relevant population of HD patients (so-called early manifest Huntington patients).

### SUMMARY OF THE INVENTION

In a first aspect, there is provided an oligonucleotide represented by SEQ ID NO:1 for use for treating, delaying, ameliorating and/or curing Huntington wherein at least a single 35 to 45 mg dose, preferably at least a single 40 mg dose of this oligonucleotide is intrathecally administered to a human patient.

In an embodiment, at least two, at least three doses of 35 to 45 mg, preferably at least two, at least three doses of 40 mg of this oligonucleotide are intrathecally and sequentially administered to a human patient.

In an embodiment, the interval between each dose is ranged from 20 to 33 days or 23 to 30 days or 25 to 29 days, preferably the interval is 28 days.

In an embodiment, the human patient is an early manifest Huntington patient.

In an embodiment, the human patient has at least 36 CAG repeats in the Huntington transcript or preferably at least 37, 38, 39, 40, 41, 42, 43, 44, 45, 46, 47, 48, 49, 50, 51, 52, 53, 54 CAG repeats in said transcript.

In an embodiment, the human patient does not have a pathogenic mutation in another polyQ disease gene, i.e., ATXN2, CACNA1A, ATXN7, TBP, AR, ATN1, ATX3, ATXN1, PPP2R2B and/or ATXN3.

In an embodiment, the oligonucleotide of the first aspect exhibits at least one of the below defined activities:
reducing or silencing or decreasing the translation rate of said mutant HTT transcript and thus the amount of corresponding mutant HTT protein,
reducing or decreasing or lowering a mutant HTT protein level.

In an embodiment, the oligonucleotide of the first aspect reduces the amount of said mutant Huntingtin protein in the cerebrospinal fluid of the human patient by at least 10, 11, 12, 13, 14, 15, 16, 17, 18, 19, 20% after at least one single 35 to 45 mg dose, preferably at least one 40 mg dose intrathecally administrated.

In an embodiment, the oligonucleotide of the first aspect reduces the amount of said mutant Huntingtin protein in the cerebrospinal fluid of the human patient by at least 10, 11, 12, 13, 14, 15, 16, 17, 18, 19, 20% after at least two 35 to 45 mg doses, preferably after at least two 40 mg doses intrathecally administrated.

In an embodiment, the oligonucleotide of the first aspect reduces the amount of said mutant Huntingtin protein in the cerebrospinal fluid of the human patient by at least 10, 11, 12, 13, 14, 15, 16, 17, 18, 19, 20, 21, 22, 23, 24, 25, 26, 27, 28, 29, 30% after at least three 35 to 45 mg doses, preferably at least three 40 mg doses intrathecally administrated.

In an embodiment, the oligonucleotide of the first aspect decreases or lowers the ratio of mutant/total HTT protein (or the ratio of mutant/wild type HTT protein) in the cerebrospinal fluid of a human patient by at least 5%, 6%, 7%, 8%, 9%, 10%, 11%, 12%, 13%, 14%, 15%, 16%, 17%, 18% m 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60% after
at least one single 35 to 45 mg dose, preferably at least one 40 mg dose intrathecally administrated,
at least two 35 to 45 mg doses, preferably at least two 40 mg doses intrathecally administrated,
at least three 35 to 45 mg doses, preferably at least three 40 mg doses intrathecally administrated, or
four 35 to 45 mg doses, preferably four 40 mg doses intrathecally administrated.

In an embodiment, the level of Neurofilament light (Nf-L) is not increased in blood and/or in the cerebrospinal fluid of the human patient after at least one 35 to 45 mg dose, preferably at least one 40 mg dose.

In an embodiment, the level of Neurofilament light (Nf-L) is decreased in blood and/or in the cerebrospinal fluid of the human patient after at least two or three 35 to 45 mg doses, preferably at least two or three 40 mg doses.

In a second aspect, there is provided a composition for use comprising an oligonucleotide of the first aspect, wherein said composition for use is for treating, delaying, ameliorating and/or curing Huntington wherein at least a single dose of 35 to 45 mg, preferably at least a single dose of 40 mg of the oligonucleotide is intrathecally administered to a human patient.

In an embodiment, the composition for use comprises at least one excipient that may further aid in enhancing the targeting and/or delivery of said composition and/or said oligonucleotide to a tissue and/or cell and/or into a tissue and/or cell of the human patient.

In an embodiment, the compositions for use comprises artificial cerebrospinalfluid as excipient.

In a third aspect, there is provided a method for treating, delaying, ameliorating and/or curing Huntington wherein at least a single dose of 35 to 45 mg, preferably at least a single dose of 40 mg of the oligonucleotide for use of the first aspect or the composition for use of the second aspect is intrathecally administered to a human patient.

### DESCRIPTION OF THE INVENTION

Various features of the aspects and embodiments of this invention are further described below. It is noted that headings used throughout this specification are to assist navigation only and should not be interpreted as definitive, and that features described in different sections may be relevant for all aspects and embodiments described herein and may thus be combined as appropriate.

In a first aspect, the invention relates to AON1 which is for use for treating, delaying, ameliorating and/or curing Huntington wherein at least a single 20 to 60 mg dose, preferably at least a single 35 to 45 mg dose, and more preferably at least a single 40 mg dose or more preferably at least a single 60 mg dose of this oligonucleotide is intrathecally administered to a human patient.

In a preferred embodiment of this first aspect, the invention relates to an oligonucleotide represented by SEQ ID NO:1 or to AON1 for use for treating, delaying, ameliorating and/or curing Huntington wherein at least a single dose of 35 to 45 mg, preferably at least a single dose of 40 mg of this oligonucleotide is intrathecally administered to a human patient.

### AON1

The oligonucleotide used herein is named AON1 and is represented by SEQ ID NO:1. Its base sequence consists of (CUG)7 wherein all its cytosines have been replaced by 5-methylcytosine. This single stranded oligonucleotide is a modified RNA and consists of 2′-O-methyl phosphorothioate RNA. AON1 has been described in WO 2013/162363 whose disclosure is incorporated by reference in its entirety. More information is given as to AON1 in the part of the definition entitled “General definitions related to oligonucleotides and to AON1”.

AON1 acts via steric hindrance of protein synthesis of mutant Huntingtin (HTT) protein. Binding of AON1 to the CAG repeat in Huntingtin (pre)messenger RNA (mRNA) transcripts sterically hinders translation and thus reduces levels of the corresponding HTT proteins, without degradation of the transcript itself (Datson N., et al., PLoS One, 2017. 12(2): p. e0171127). AON1 generally reduces levels of WT Huntingtin to a lesser extent than mutant Huntingtin, a phenomenon referred to as allelic preference. Factors likely affecting the degree of allelic preference include the length of the CAG repeat, the length difference with the corresponding WT repeat, and the interruptions within the CAG repeat that may affect binding efficacy of AON1.

Other mechanisms that have been proposed to play a potential role in HD pathogenesis and could be prevented/reduced by AON1 include RNA toxicity (Rue L. et al., 2016, J Clin Invest., 126(11):4319-4330), RAN translation (Bañez-Coronel M. et al., 2015, Neuron, 88(4):667-77), somatic CAG repeat expansion (Kennedy L. et al. 2003, Human Molecular Genetics, 12(24):3359-3367) and formation of toxic exon 1 by aberrant splicing (Sathasivam K. et al., 2013, Proc Natl Acad Sci USA, 110(6):2366-70).

Therefore, in a preferred embodiment, AON1 is expected to exhibit at least one of the below defined activities:
reducing or silencing or decreasing the translation rate of said mutant HTT transcript and thus the amount of the corresponding mutant HTT protein,
reducing or decreasing a mutant HTT protein level,
binding to the CAG repeat in said mutant HTT transcript and sterically hindering any resulting RNA toxicity of the repeat itself,
reducing or silencing or decreasing repeat-associated non-ATG (RNA) translation of the CAG repeat and thus the corresponding amount of homoploymeric proteins polyGln, polyAla, polySer, polyCys and polyLeu),
binding to ultra long (>100->1000) CAG repeats in said mutant HTT transcript that are the result of somatic expansion and reducing or decreasing translation into an ultra long polyQ stretch in the corresponding mutant HTT protein level,
binding to the CAG repeat in said mutant HTT transcript and preventing/reducing induction of aberrant splicing of HTT exon 1, thus lowering levels of a short polyadenylated mRNA that is translated into a toxic exon 1 HTT protein and thus lowering levels of said toxic exon 1 HTT protein.
decreasing or lowering the ratio of mutant/total HTT protein (or the ratio of mutant/wild type HTT protein).

More preferably, AON1 is expected to exhibit at least one of the below defined activities:
reducing or silencing or decreasing the translation rate of said mutant HTT transcript and thus the amount of the corresponding mutant HTT protein and
reducing or decreasing mutant HTT protein levels.

More preferably, AON1 is expected to exhibit at least one of the below defined activities:
reducing or silencing or decreasing the translation rate of said mutant HTT transcript and thus the amount of the corresponding mutant HTT protein,
reducing or decreasing mutant HTT protein levels and
decreasing or lowering the ratio of mutant/total HTT protein (or the ratio of mutant/wild type HTT protein).

More preferably, AON1 is expected to exhibit the below defined activity: decreasing or lowering the ratio of mutant/total HTT protein (or the ratio of mutant/wild type HTT protein).

Moreover, in another preferred embodiment, AON1 is expected to exhibit one of the below defined additional effects on another marker, which is the Neurofilament light (Nf-L):
the level of Neurofilament light (Nf-L) is not increased in blood and/or in the cerebrospinal fluid of the human patient.
the level of Neurofilament light (Nf-L) is reduced or decreased or lowered in blood and/or in the cerebrospinal fluid of the human patient.

Alternatively or in combination with previous preferred embodiment, in the context of the invention, AON1 may be able to treat, delay, ameliorate and/or a human genetic disorder as Huntington's disease (HD), when AON1 is able to alleviate one or more symptom(s) and/or characteristic(s) and/or to improve a parameter linked with or associated with Huntington's disease (HD) in an individual as later defined herein.

These activities may be assessed in a cell of a patient, in a tissue of a patient and/or in a patient as explained later herein.

In the context of the invention, a mutant HTT transcript encodes a mutant HTT protein. A mutant HTT transcript or mutant HTT protein may also be called a disease-associated or disease-causing transcript (or protein) or toxic transcript (or protein). Such mutant HTT transcript contains an extended or unstable number of repeats in a cell of a patient, in a tissue of a patient and/or in a patient as explained later herein.

AON1 is for use as a medicament for treating, delaying, ameliorating and/or curing Huntington and is considered to exhibit such therapeutic activity as soon as one of the above defined activities has been demonstrated and as further explained below.

In an embodiment, AON1 is considered to reduce or silence or decrease the translation rate of said mutant HTT transcript and thus the amount of the mutant HTT protein in a cell of said patient, in a tissue of said patient and/or in a patient when the reduction or decrease of the translation rate of said transcript or of said mutant HTT protein is at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the translation rate of said transcript before the treatment or at the onset of the treatment.

In another embodiment, AON1 reduces or decreases or lowers the mutant HTT protein level when the mutant HTT protein level is reduced or decreased or lowered at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the mutant HTT protein level before the treatment or at the onset of the treatment.

In an embodiment, AON1 reduces or decreases or lowers the ratio of mutant/total HTT protein (or the ratio of mutant/wild type HTT protein) in the cerebrospinal fluid of a human patient by at least 5%, 6%, 7%, 8%, 9%, 10%, 11%, 12%, 13%, 14%, 15%, 16%, 17%, 18% m 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%.

These reductions or decreases of the translation rate of said transcript or these reductions or decreases or lowerings of the mutant protein level may be assessed in a cell of said patient, in a tissue of said patient and/or in a patient. In a preferred embodiment, the assessment is made in the cerebrospinal fluid of the patient. In another preferred embodiment, the assessment is made in the blood, preferably the plasma of the patient.

The onset of the treatment may mean the day wherein the first dose is given to a patient.

In an embodiment, AON1 decreases or lowers the ratio of mutant/total HTT protein (or the ratio of mutant/wild type HTT protein) in the cerebrospinal fluid of a human patient by at least 5%, 6%, 7%, 8%, 9%, 10%, 11%, 12%, 13%, 14%, 15%, 16%, 17%, 18% m 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60% after
at least one single 35 to 45 mg dose intrathecally administrated, preferably at least one 40 mg dose intrathecally administrated,
at least two 35 to 45 mg doses intrathecally administrated, preferably at least two 40 mg doses intrathecally administrated,
at least three 35 to 45 mg doses intrathecally administrated, preferably at least three 40 mg doses intrathecally administrated,
four 35 to 45 mg doses intrathecally administrated, preferably four 40 mg doses intrathecally administrated.
at least one single 20 to 60 mg dose intrathecally administrated, preferably at least one 40 mg dose intrathecally administrated,
at least two single 20 to 60 mg dose intrathecally administrated, preferably at least two 40 mg dose intrathecally administrated,
at least three single 20 to 60 mg dose intrathecally administrated, preferably at least three 40 mg dose intrathecally administrated or
at least four single 20 to 60 mg dose intrathecally administrated, preferably at least four 40 mg dose intrathecally administrated.

In a preferred embodiment, this decrease or lowering is seen after one to four 40 mg dose after at least one week, at least two weeks, at least three weeks, and/or at least three weeks and is still visible after at least 20, 40, 60, 80 days after the first administration of AON1. This has been demonstrated in FIG. 6.

The use of the ratio of mutant/total HTT protein (or the ratio of mutant/wild type HTT protein) is a new and sensitive way of monitoring the selective activity of AON1 on the mutated allele of the HTT protein. The relevancy of these ratio has been demonstrated in FIG. 6.

In another embodiment, the level of Neurofilament light (Nf-L) is not increased (or is preferably decreased or reduced or lowered) in blood and/or in the cerebrospinal fluid of the human patient after treatment with AON1. The decrease of the level of Nf-L may be at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the level of Nf-L before treatment or at the onset of the treatment. In an embodiment, the assessment is made in the blood, preferably the plasma of the patient. The onset of the treatment may mean the day wherein the first dose is given to a patient.

These reductions or decreases may be assessed by highly sensitive and specific immunoassays including ELISA and single molecule counting (SMC). Mutant HTT can be assessed in CSF by immunoassays with a femtomolar detection threshold, and a broad dynamic range, such as the ultrasensitive SMC method on the SMCxPRO platform using antibodies 2B7 (anti-HTT(7-13) mouse monoclonal antibody) and MW1 (anti-poly-Q mouse monoclonal antibody) (Wild E. et al., 2015, J Clin Invest, 125(5):1979-86). Nf-L levels in CSF can be quantified using a digital immunoassay (Nf-L Simoa NF-light V2 Advantage Kit on the Quanterix SR-X platform).

### Dose(s) and Therapeutic Effect

The inventors are the first to have demonstrated that a single dose of AON1 ranged from 35 to 45 mg was able to exhibit a therapeutic effect in HD patients. This is surprising and unexpected. AON1 is intrathecally administrated to the patient.

In en embodiment, the single dose is ranged from 20 to 60 mg. The single dose may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mg.

Therefore in one embodiment, AON1 is for use for treating, delaying, ameliorating and/or curing Huntington wherein at least a single 20 to 60 mg dose, preferably at least a single 35 to 45 mg dose, and more preferably at least a single 40 mg dose or more preferably at least a single 60 mg dose of this oligonucleotide is intrathecally administered to a human patient.

In a preferred embodiment, the single dose may be 35, 36, 37, 38, 39, 40, 41, 42, 43, 44 or 45 mg. More preferably the single dose is 38, 39, 40, 41 or 42 mg. Even more preferably the dose is 40 mg.

As demonstrated in the experimental part, a single 40 mg dose of AON1 is able to decrease the level of mutant HTT in the cerebrospinal fluid of patients of at least 15% 28 days after the administration of AON1 (see FIG. 4). The decrease may be of at least 5, 6, 7, 8, 9, 10, 11, 12, 13, 14, 15, 16, 17, 18%.

Therefore in an embodiment, AON1 is for use for treating, delaying, ameliorating and/or curing Huntington reduces the amount of said mutant Huntingtin protein in the cerebrospinal fluid of the human patient by at least 10, 11, 12, 13, 14, 15, 16, 17, 18, 19, 20% after at least one single 20 to 60 mg dose, preferably after at least one single 35 to 45 mg dose, and more preferably after at least one 40 mg dose or more preferably after at least one single 60 mg dose intrathecally administrated. Therefore, in a preferred embodiment, AON1 reduces the amount of said mutant Huntingtin protein in the cerebrospinal fluid of the human patient by at least 5, 6, 7, 8, 9, 10, 11, 12, 13, 14, 15, 16, 17, 18, 19, 20% after at least one single 35 to 45 mg dose of AON1, preferably after at least one single 40 mg dose of intrathecally administrated AON1.

Moreover, the inventors are the first to have demonstrated that the therapeutic effect observed after one single dose remains visible or is even improved after two doses of AON1 ranged from 35 to 45 mg. AON1 is also intrathecally administrated to the patient.

In one embodiment, AON1 is for use for treating, delaying, ameliorating and/or curing Huntington as follows: at least two doses of 20 to 40 mg, preferably at least two doses of 35 to 45 mg, more preferably at least two doses of 40 mg or more preferably at least two doses of 20 mg of this oligonucleotide are intrathecally and sequentially administered to a human patient. Each of the two doses may be 20, 21, 22, 23, 24, 25, 26, 27, 28, 29, 30, 31, 32, 33, 34, 35, 36, 37, 38, 39, 40, 41, 42, 43, 44, 45.

In a preferred embodiment, each of the two doses may be 35, 36, 37, 38, 39, 40, 41, 42, 43, 44 or 45 mg. More preferably each of the two doses is 38, 39, 40, 41 or 42 mg. Even more preferably each of the two doses is 40 mg.

As demonstrated in the experimental part, two 40 mg doses of AON1 are able to decrease the level of mutant HTT in the cerebrospinal fluid of patients of at least 15% 28 days after the administration of the second dose of AON1 (see FIG. 4). The decrease may be of at least 5, 6, 7, 8, 9, 10, 11, 12, 13, 14, 15, 16, 17, 18%.

Therefore in one embodiment, AON1 for use for treating, delaying, ameliorating and/or curing Huntington, wherein said oligonucleotide reduces the amount of said mutant Huntingtin protein in the cerebrospinal fluid of the human patient by at least 10, 11, 12, 13, 14, 15, 16, 17, 18, 19, 20% after at least two 20 to 40 mg doses, preferably at least two 35 to 45 mg doses, more preferably after at least two 40 mg doses intrathecally administrated.

In a preferred embodiment, AON1 reduces the amount of said mutant Huntingtin protein in the cerebrospinal fluid of the human patient by at least 5, 6, 7, 8, 9, 10, 11, 12, 13, 14, 15, 16, 17, 18, 19, 20% after at least two 35 to 45 mg doses of AON1, preferably at least two 40 mg dose of AON1 intrathecally administrated.

Moreover, the inventors are the first to have demonstrated that the therapeutic effect observed after two doses remains visible or is even improved. The same holds after three doses of AON1.

In an embodiment, the same holds after three doses of AON1 ranged from 20 to 40 mg. Each of the three doses may be 20, 21, 22, 23, 24, 25, 26, 27, 28, 29, 30, 31, 32, 33, 34, 35, 36, 37, 38, 39, 40, 41, 42, 43, 44, 45.

In an embodiment, AON1 is for use for treating, delaying, ameliorating and/or curing Huntington as follows: at least three doses of 20 to 40 mg, preferably at least three doses of 35 to 45 mg, more preferably at least three doses of 40 mg or more preferably at least three doses of 20 mg of this oligonucleotide are intrathecally and sequentially administered to a human patient.

In a preferred embodiment, the three doses of AON1 are ranged from 35 to 45 mg. AON1 is intrathecally administrated to the patient. Each of the three doses may be 35, 36, 37, 38, 39, 40, 41, 42, 43, 44 or 45 mg. Preferably each of the three doses is 38, 39, 40, 41 or 42 mg. More preferably each of the three doses is 40 mg.

As demonstrated in the experimental part, three 40 mg doses of AON1 are able to decrease the level of mutated HTT in the cerebrospinal fluid of patients of at least 28% 28 days after the administration of the third dose of AON1 (see FIG. 4). The decrease may be of at least 5, 6, 7, 8, 9, 10, 11, 12, 13, 14, 15, 16, 17, 18, 19, 20, 21, 22, 23, 24, 25, 26, 27, 28, 29, 30%.

In an embodiment, AON1 reduces the amount of said mutant Huntingtin protein in the cerebrospinal fluid of the human patient by at least 10, 11, 12, 13, 14, 15, 16, 17, 18, 19, 20, 21, 22, 23, 24, 25, 26, 27, 28, 29, 30% after at least three 20 to 40 mg doses, preferably at least three 35 to 45 mg doses, more preferably at least three 40 mg doses or more preferably at least three 20 mg doses intrathecally administrated.

In a preferred embodiment, AON1 reduces the amount of said mutant Huntingtin protein in the cerebrospinal fluid of the human patient by at least 10, 11, 12, 13, 14, 15, 16, 17, 18, 19, 20, 21, 22, 23, 24, 25, 26, 27, 28, 29, 30% after at least three 35 to 45 mg doses, preferably after at least three 40 mg doses intrathecally administrated.

Last but not least, the inventors are the first to have further corroborated the therapeutic effect of AON1 observed on the decrease of mutant HTT protein by a positive effect on the critical biomarker associated with neurodegeneration and neurological conditions: Neurofilament light protein (Nf-L) (FIG. 5).

In an embodiment, the level of Neurofilament light (Nf-L) is not increased in blood and/or in the cerebrospinal fluid of the human patient after at least one 20 to 60 mg dose, preferably at least one 35 to 45 mg dose, more preferably at least one 40 mg dose or more preferably at least one 60 mg dose. AON1 is intrathecally administrated. In a preferred embodiment, the level of Neurofilament light (Nf-L) is not increased in blood and/or in the cerebrospinal fluid of the human patient after at least one 35 to 45 mg dose, preferably at least one 40 mg dose of AON1. AON1 is intrathecally administrated.

In an embodiment, the level of Neurofilament light (Nf-L) is decreased in blood and/or in the cerebrospinal fluid of the human patient after at least one 35 to 45 mg dose, preferably at least one 40 mg dose of AON1. The decrease may be of at least 1, 2, 3, 4, 5, 6, 7, 8, 9, 10%.

In an embodiment, the level of Neurofilament light (Nf-L) is decreased in blood and/or in the cerebrospinal fluid of the human patient after at least two or three 20 to 40 mg doses, preferably at lest two or three 35 to 45 mg doses, and more preferably at least two or three 40 mg doses or more preferably at least or three 20 mg doses. The decrease may be of at least 1, 2, 3, 4, 5, 6, 7, 8, 9, 10%. AON1 is intrathecally administrated. The interval between each dose may be as later disclosed herein.

In a preferred embodiment, the level of Neurofilament light (Nf-L) is decreased in blood and/or in the cerebrospinal fluid of the human patient after at least two or three 35 to 45 mg doses, preferably at least two or three 40 mg doses of AON1. The decrease may be of at least 1, 2, 3, 4, 5, 6, 7, 8, 9, 10%. AON1 is intrathecally administrated. The interval between each dose may be as later disclosed herein.

Nf-L may be assessed by digital immunoassay for the quantitative determination of NF-L in serum, plasma and CSF, such as the Simoa NF-light V2 Advantage Kit (Quanterix).

In en embodiment, at least one or at least two or at least three doses of 20 to 60 mg of AON1 are administrated to HD patients. The single dose may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mg. The single dose may be 20 mg. The single dose may be 40 mg. The single dose may be 60 mg.

Each of the two doses may be 35, 36, 37, 38, 39, 40, 41, 42, 43, 44, 45 mg. Each of the two doses may be 20 mg. Each of the two doses may be 40 mg. Each of the two doses may be 60 mg.

Each of the three doses may be 20, 21, 22, 23, 24, 25, 26, 27, 28, 29, 30, 31, 32, 33, 34, 35, 36, 37, 38, 39, 40, 41, 42, 43, 44, 45 mg. Each of the three doses may be 20 mg. Each of the three doses may be 40 mg.

In a preferred embodiment, at least one, or at least two, or at least three doses of 35 to 45 mg of AON1 are administrated to HD patients. The single dose may be 35, 36, 37, 38, 39, 40, 41, 42, 43, 44 or 45 mg. Preferably the single dose is 38, 39, 40, 41 or 42 mg. More preferably the single dose is 40 mg.

In an embodiment, at least two doses of AON1 are administrated. Each of the two doses may be 35, 36, 37, 38, 39, 40, 41, 42, 43, 44 or 45 mg. Preferably each of the two doses is 38, 39, 40, 41 or 42 mg. More preferably each of the two doses is 40 mg.

In an embodiment, at least three doses of AON1 are administrated. Each of the three doses may be 35, 36, 37, 38, 39, 40, 41, 42, 43, 44 or 45 mg. Preferably each of the three doses is 38, 39, 40, 41 or 42 mg. More preferably each of the three doses is 40 mg.

In an embodiment, the doses are administrated in an infrequent dosing schedule and are quite spaced in time due to an estimated half life of 150-200 days in brain tissues.

In an embodiment, the doses are sequentially administrated with an interval between each dose which is ranged from 20 days to 1 year or from 20 days to 11 months or from 20 days to 10 months or from 20 days to 9 months or from 20 days to 8 months or from 20 days to 7 months or from 20 days to 6 months or from 20 days to 5 months or from 20 days to 4 months or from 20 days to 3 months or from 20 days to 2 months or from 20 days to 1 month. In an embodiment, the doses are sequentially administrated every 1, 2, 3, 4, 5, 6, 7, 8 months. In a preferred embodiment, the doses are sequentially administered every 6, 7 or 8 months.

The doses are as defined earlier herein, from 20 to 60 mg, preferably from 35 to 45 mg and more preferably 38, 39, 40, 41 or 42 mg. In an embodiment, each dose is 20 mg. In an embodiment, each dose is 40 mg. In an embodiment, each dose is 60 mg. More preferably each dose is 40 mg. In a preferred embodiment, the administration is intrathecal.

In an embodiment, the doses are sequentially administered with an interval between each dose ranging from:
1) 20 to 33 days or 23 to 30 days or 25 to 29 days, preferably the interval is 25, 26, 27, 28, or 29 days, more preferably 28 days or
2) 34 to 200 days or 50 to 180 days or 60 to 170 days or 70 to 169 days or 80 to 169 days or 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169 preferably the interval is 85 to 169 days or preferably the interval is 84 to 168 days or preferably the interval is 84 days.

The doses are as defined earlier herein, from 20 to 60 mg, preferably from 35 to 45 mg and more preferably 38, 39, 40, 41 or 42 mg. In an embodiment, each dose is 20 mg. In an embodiment, each dose is 40 mg. In an embodiment, each dose is 60 mg. More preferably each dose is 40 mg.

In an embodiment, one single 60 mg dose is administrated.

In another embodiment, two 40 mg doses are administrated with an interval of 120 to 200 days or 140 to 190 days or 150 to 180 days or 160 to 170 days or 169 days or 168 days.

In another embodiment, three 20 mg doses are administrated with an interval of 30 to 200 days or 50 to 190 days or 60 to 150 days or 70 to 100 days or 80 to 90 days or 85 days or 84 days.

In an embodiment, a 20 mg dose is administrated three times. In an embodiment the second dose is administrated 80, 81, 82, 83, 84, 84, 85, 86, 87, 88, 89, 90 days after the first dose. In an embodiment, the third dose is administrated 160, 161, 162, 163, 164, 165, 166, 167, 168, 169, 170, 171, 172, 173, 174, 175, 176, 177, 178, 179 or 180 days after the first dose.

In a preferred embodiment, a 20 mg dose is administrated at day 1, day 85 and day 169.

In a preferred embodiment, the administration is intrathecal.

In an embodiment, the doses are sequentially administrated with an interval between each dose which is ranged from 20 to 33 days or 23 to 30 days or 25 to 29 days, the interval may be 20, 21, 22, 23, 24, 25, 26, 27, 28, 29, 30, 31, 32, 33 days, preferably the interval is 28 days. The doses are as defined earlier herein, from 20 to 60 mg, preferably from 35 to 45 mg and more preferably 38, 39, 40, 41 or 42 mg. In an embodiment, each dose is 20 mg. In an embodiment, each dose is 40 mg. In an embodiment, each dose is 60 mg. More preferably each dose is 40 mg. In a preferred embodiment, the administration is intrathecal.

### Patient Population

Within the context of the invention, AON1 may be administered to a subject or a patient, such as a subject in need thereof. In some embodiments, the subject (in need) can be a healthy, asymptomatic or partially symptomatic subject (for example before (full) onset of the disease). In some embodiments, the subject (in need) may suffer from or any of the symptoms thereof, or be at risk for developing Huntington's disease (HD). In some embodiments, the subject (in need) may be a subject inflicted with Huntington's disease (HD).

A repeat or repetitive element or repetitive sequence or repetitive stretch is herein defined as a repetition of at least 3, 4, 5, 10, 20, 30, 40, 50, 60, 70, 80, 90, 100, 1000 or more, of the repetitive unit or repetitive nucleotide unit or repeat nucleotide unit (CAG)n comprising a trinucleotide repetitive unit CAG in a transcribed HTT gene sequence in the genome of a subject, including a human subject. Accordingly, n is an integer and may be at least 3, 4, 5, 10, 20, 30, 40, 50, 60, 70, 80, 90, 100, 1000 or more. In the majority of patients, a “pure” repeat or repetitive element or repetitive sequence or repetitive stretch as identified above ((CAG)n,) is present in a transcribed HTT gene sequence in the genome of said patient. However, it is also encompassed by the invention, that in some patients, the penultimate CAA codon downstream of said repeat or repetitive element or repetitive sequence or repetitive stretch as identified above is qualified as a “variant” when for example said repeat or repetitive element or repetitive sequence or repetitive stretch as identified above is extended by complete loss of interrupting (LOI) adenine nucleotides in this region (Wright GEB et al. 2019, Am J Hum Genet 104(6):1116-1126).

Such (CAG)n repeat or repetitive element or repetitive sequence or repetitive stretch is present in exon 1 of the HTT gene in the genome of a patient. Such repeat may be defined herein as comprising a consecutive repetition of 36 or more repetitive units comprising a CAG trinucleotide, in a transcribed sequence of the HTT gene.

Throughout the invention, the term CAG repeat may be replaced by (CAG)n, and vice versa, wherein n is an integer that may be up to 35 when the repeat is present in exon 1 of the HTT transcript of a healthy individual.

HD classically manifests with a triad of motor, cognitive and behavioural features (Folstein S E., et al, 1991, Res Publ Assoc Res Nerv Ment Dis 69: 181-191). During prodromal period, patients may experience variable cognitive and behavioural changes with subtle motor changes, which are not sufficient to confirm the diagnosis of HD. The manifest disease, typically lasts for 10-20 years, is characterised by motor and cognitive changes that progress inexorably over the course of the illness until death.

The manifest disease period can be subdivided into five stages based on evolving changes in motor symptoms and functional capacity (Ross C A., et al, 2014, Nat Rev Neurol, 10(4): p 204-216). Stage I represents the highest level of capacity and is characterised by mild or no incapacity in terms of independence in daily activities, managing personal finances and ability to maintain employment, while Stage V represents severe disability and dependence on full-time care (Shoulson I. et al., 1979, Neurology, 29: 1-3).

The five stages also correlate with score on Unified Huntington's Disease Rating Scale (UHDRS) including the Total Functional Capacity (TFC) Scale, with Stage I corresponding to TFC scores of 11-13 (least severe); Stage II to scores of 7-10; Stage III to scores of 3-6; Stage IV to scores of 1-2; Stage V to a score of 0 (most severe).

The cUHDRS is a multidomain measure encompassing motor, functional, and cognitive scales, all of which are independently associated with HD severity (Estevez-Fraga C. et al., 2021, Movement Disorders 36(5):1259-1264).

The TFC uses a validated scale or symptom progression regarding the three main symptomatic areas of HD, measured by validated rating scales (Shoulson I et al., 1979, Neurology, 29:1-3). These areas are specifically progression of motor signs, progression of neuropsychiatric symptoms and progression of cognitive decline.

In a preferred embodiment, human HD patients are early manifest HD human patient. In an embodiment the onset of the disease is at the adult age. In an embodiment, the UHDRS is used to classify the patient. In another embodiment, the composite UHDRS (cUHDRS) is used to classify the patient. In another embodiment, the total functional capacity (TFC) is used to classify the patient (

In a more preferred embodiment, patients are classified using the TFC, the UHDRS and the CAG repeat length.

Early manifest HD (Stages I and II with TFC scores of 7-13) is generally characterised by involuntary movements of the face, fingers, feet or thorax (Folstein S E et al, 1991, Res Publ Assoc Res Nerv Ment Dis 69: 181-191). The assessment of the diagnosis of an early manifest patient is done by a physician. Usually, this assessment is done using TFC scores.

In another preferred embodiment, the human patient has at least 36 CAG repeats in the Huntingtin transcript at the onset of the treatment or preferably at least 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 CAG repeats in said transcript at the onset of the treatment. In a more preferred embodiment, the number of CAG repeats in the HTT transcript is at least 36. In another preferred embodiment, the number of CAG repeats in the HTT transcript is at least 40, 41, 42, 43, 44, 45, 46, 47, 48, 49, 50, 51, 52, 53 or 54. In another preferred embodiment, the number of CAG repeats in the HTT transcript is 45, 46, 47, 48, 49, 50 or 51. The number of repeats is assessed by sequencing the HTT transcript or the HTT gene of the patient.

In another embodiment, the human patient does not have a pathogenic mutation in another polyQ disease gene, such as ATXN1, ATXN2, ATXN3, CACNA1A, PPP2R2B, ATXN7, TBP, AR and ATN1.

Such pathogenic mutation may be defined as: ≥41 contiguous, uninterrupted CAG repeats in ATXN1; ≥33 CAG repeats in ATXN2; ≥61 repeats in ATXN3; ≥20 CAG repeats in CACNA1A; ≥34 CAG repeats in ATXN 7; ≥41 CAG repeats in TBP; 43 CAG repeats in PPP2R2B; ≥38 CAG repeats in AR; ≥48 CAG repeats in ATN1. The assessment of the presence of a pathogenic mutation in another polyQ disease gene is carried out by sequencing the transcript (premRNA) or the corresponding gene. The sequences of all human healthy transcripts or complementary DNA (cDNA) are identified as: HTT (SEQ ID NO: 2), ATXN1 (SEQ ID NO:3), ATXN2 (SEQ ID NO: 4) ATXN3 (SEQ ID NO: 5), CACNA1A (SEQ ID NO:6), ATXN7 (SEQ ID NO: 7), PPP2R2B (SEQ ID NO: 8), TBP (SEQ ID NO:9), AR (SEQ ID NO: 10) or ATN1 (SEQ ID NO: 11).

### Other Parameters/Symptoms of HD

AON1 may be able to improve at least another parameter or reduce a symptom or characteristic of a HD patient treated under the conditions described earlier herein.

Improvement in this context may mean that said parameter had been significantly changed towards a value of said parameter for a healthy person and/or towards a value of said parameter that corresponds to the value of said parameter in the same individual at the onset of the treatment.

Reduction or alleviation in this context may mean that said symptom or characteristic had been significantly changed towards the absence of said symptom or characteristic which is characteristic for a healthy person and/or towards a change of said symptom or characteristic that corresponds to the state of the same individual at the onset of the treatment.

In this context, symptoms for Huntington's Disease are choreiform movements, progressive dementia and psychiatric manifestations (depression, psychosis, etc.). Choreiform movements consist of involuntary, rapid, irregular, jerky motor actions including facial twitching or writhing and twitching of distal extremities, and later more generalized forms that may impair gait (Roos R A. 2010, Orphanet. J. Rare Dis., 5, 40)). Each of these symptoms may be assessed by the physician using known and described methods.

Another parameter may be mutant HTT aggregates that are found in the nucleus and cytoplasm of cells, indicating that protein misfolding is a common feature of HD. Another parameter is therefore aggregated mutant HTT protein in the nucleus and/or cytoplasm. Such aggregates may be monitored by in situ hybridization. An improvement of such a HD parameter may be the decrease in the detection of such protein aggregate. Such decrease may be at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the quantity or amount of protein aggregate before the onset of the treatment.

### Composition for Use

In a further aspect, there is provided a composition comprising an oligonucleotide as described in the previous section entitled “AON1”. This composition preferably comprises or consists of or essentially consists of AON1 as described above.

Features relating to AON1 have been disclosed in the section entitled “AON1” and in the definition section entitled “General definitions related to oligonucleotides and to AON1.

In a preferred embodiment, said composition is for use as a medicament. Said composition is therefore a pharmaceutical composition. A pharmaceutical composition usually comprises a pharmaceutically accepted carrier, diluent and/or excipient.

In an embodiment, there is provided a composition for use comprising AON1 for use as defined earlier herein, wherein said composition for use is for treating, delaying, ameliorating and/or curing Huntington wherein at least a single dose of 20 to 60 mg, preferably at least a single dose of 35 to 45 mg, and more preferably at least a single dose of 40 mg or more preferably at least a single dose of 60 mg of the oligonucleotide is intrathecally administered to a human patient. Features of AON1 and of its uses also hold for a composition comprising AON1 and have already been defined in the section entitled “AON1” and in the definition section entitled “General definitions related to oligonucleotides and to AON1”.

In a more preferred embodiment, there is provided a composition for use comprising AON1 for use as earlier defined herein, wherein said composition for use is for treating, delaying, ameliorating and/or curing Huntington wherein at least a single dose of 35 to 45 mg, preferably at least a single 40 mg dose of the oligonucleotide is intrathecally administered to a human patient. Features of AON1 and of its uses also hold for a composition comprising AON1 and have already been defined in the section entitled “AON1” and in the definition section entitled “General definitions related to oligonucleotides and to AON1”.

In an embodiment, a composition of the current invention comprises a compound (i.e. an oligonucleotide) as defined herein and optionally further comprises a pharmaceutically acceptable formulation, filler, preservative, solubilizer, carrier, diluent, excipient, salt, adjuvant and/or solvent. Such pharmaceutically acceptable carrier, filler, preservative, solubilizer, diluent, salt, adjuvant, solvent and/or excipient may for instance be found in Remington: The Science and Practice of Pharmacy, 20th Edition. Baltimore, MD: Lippincott Williams & Wilkins, 2000. The compound as described in the invention possesses at least one ionizable group. An ionizable group may be a base or acid, and may be charged or neutral. An ionizable group may be present as ion pair with an appropriate counterion that carries opposite charge(s). Examples of cationic counterions are sodium, potassium, cesium, Tris, lithium, calcium, magnesium, trialkylammonium, triethylammonium, and tetraalkylammonium. Examples of anionic counterions are chloride, bromide, iodide, lactate, mesylate, acetate, trifluoroacetate, dichloroacetate, and citrate. Examples of counterions have been described [e.g. Kumar L. et al, 2008, which is incorporated here in its entirety by reference].

A pharmaceutical composition may be further formulated to further aid in enhancing the stability, solubility, absorption, bioavailability, pharmacokinetics and cellular uptake of said compound, in particular formulations comprising excipients capable of forming complexes, nanoparticles, microparticles, nanotubes, nanogels, hydrogels, poloxamers or pluronics, polymersomes, colloids, microbubbles, vesicles, micelles, lipoplexes, and/or liposomes. Examples of nanoparticles include polymeric nanoparticles, gold nanoparticles, magnetic nanoparticles, silica nanoparticles, lipid nanoparticles, sugar particles, protein nanoparticles and peptide nanoparticles.

A preferred composition comprises at least one excipient that may further aid in enhancing the targeting and/or delivery of said composition and/or said oligonucleotide to and/or into brain tissue and/or to a neuronal tissue and/or a cell. A cell may be a neuronal cell.

In an embodiment, a composition for use comprises at least one excipient that may further aid in enhancing the targeting and/or delivery of said composition and/or said oligonucleotide AON1 to a tissue and/or cell and/or into a tissue and/or cell of the human patient.

In a preferred embodiment, a composition comprises AON1 and artificial cerebrospinal fluid as excipient. Artificial cerebrospinal fluid is a fluid that mimics the characteristics of human cerebrospinal fluid. Typically it contains 150 mM Na, 3.0 mM K, 1.4 mM Ca, 0.8 mM Mg, 1.0 mM P, 155 mM Cl (Davson, H. Physiology of the Cerebrospinal Fluid, J. & A. Churchill, Ltd., London, 1967 and Biology Data Book, Volume III, 2nd ed., Fed. Am. Soc. Exper. Biol., Washington D.C., 1974).

Other excipients are known in the art (e.g. see Bruno, 2011) and may be categorized as a first type of excipient. Examples of first type of excipients include polymers (e.g. polyethyleneimine (PEI), polypropyleneimine (PPI), dextran derivatives, butylcyanoacrylate (PBCA), hexylcyanoacrylate (PHCA), poly(lactic-co-glycolic acid) (PLGA), polyamines (e.g. spermine, spermidine, putrescine, cadaverine), chitosan, poly(amido amines) (PAMAM), poly(ester amine), polyvinyl ether, polyvinyl pyrrolidone (PVP), polyethylene glycol (PEG) cyclodextrins, hyaluronic acid, colominic acid, and derivatives thereof), dendrimers (e.g. poly(amidoamine)), lipids {e.g. 1,2-dioleoyl-3-dimethylammonium propane (DODAP), dioleoyldimethylammonium chloride (DODAC), phosphatidylcholine derivatives [e.g 1,2-distearoyl-sn-glycero-3-phosphocholine (DSPC)], lyso-phosphatidylcholine derivaties [e.g. 1-stearoyl-2-lyso-sn-glycero-3-phosphocholine (S-LysoPC)], sphingomyeline, 2-{3-[bis-(3-amino-propyl)-amino]-propylamino}-N-ditetracedyl carbamoyl methylacetamide (RPR209120), phosphoglycerol derivatives [e.g. 1,2-dipalmitoyl-sn-glycero-3-phosphoglycerol sodium salt (DPPG-Na), phosphaticid acid derivatives [1,2-distearoyl-sn-glycero-3-phosphaticid acid, sodium salt (DSPA), phosphatidylethanolamine derivatives [e.g. dioleoyl-phosphatidylethanolamine (DOPE), 1,2-distearoyl-sn-glycero-3-phosphoethanolamine (DSPE),2-diphytanoyl-sn-glycero-3-phosphoethanolamine (DPhyPE),], N-[1-(2,3-dioleoyloxy)propyl]-N,N,N-trimethylammonium (DOTAP), N-[1-(2,3-dioleyloxy)propyl]-N,N,N-trimethylammonium (DOTMA), 1,3-di-oleoyloxy-2-(6-carboxy-spermyl)-propylamid (DOSPER), (1,2-dimyristyolxypropyl-3-dimethylhydroxy ethyl ammonium (DMRIE), (N1-cholesteryloxycarbonyl-3,7-diazanonane-1,9-diamine (CDAN), dimethyldioctadecylammonium bromide (DDAB), 1-palmitoyl-2-oleoyl-sn-glycerol-3-phosphocholine (POPC), (b-L-arginyl-2,3-L-diaminopropionic acid-N-palmityl-N-olelyl-amide trihydrochloride (AtuFECT01), N,N-dimethyl-3-aminopropane derivatives [e.g. 1,2-distearoyloxy-N,N-dimethyl-3-aminopropane (DSDMA), 1,2-dioleyloxy-N,N-dimethyl-3-aminopropane (DoDMA), 1,2-dilinoleyloxy-N,N-3-dimethylaminopropane (DLinDMA), 2,2-dilinoleyl-4-dimethylaminomethyl [1,3]-dioxolane (DLin-K-DMA), phosphatidylserine derivatives [1,2-dioleyl-sn-glycero-3-phospho-L-serine, sodium salt (DOPS)], cholesterol}proteins (e.g. albumin, gelatins, atellocollagen), and peptides (e.g. protamine, PepFects, NickFects, polyarginine, polylysine, CADY, MPG).

Another preferred composition may comprise at least one excipient categorized as a second type of excipient. A second type of excipient may comprise or contain a conjugate group as described herein to enhance targeting and/or delivery of the composition and/or of the oligonucleotide of the invention to a tissue and/or cell and/or into a tissue and/or cell, as for example neuronal tissue. Both types of excipients may be combined together into one single composition as identified herein.

The skilled person may select, combine and/or adapt one or more of the above or other alternative excipients and delivery systems to formulate and deliver a compound for use in the present invention.

Such a pharmaceutical composition of the invention may be administered in an effective concentration at set times to an animal, preferably a mammal. More preferred mammal is a human being. AON1 for use or a composition comprising AON1 for use according to the invention may be suitable for direct administration to a cell, tissue and/or an organ in vivo of individuals affected by or at risk of developing a disease or condition as identified herein, and may be administered directly in vivo, ex vivo or in vitro. Administration may be via systemic and/or parenteral routes, for example intravenous, subcutaneous, intraventricular, intrathecal, intramuscular, intranasal, enteral, intravitreal, intracerebral, epidural or oral route. A preferred administration route is intrathecal.

### Use

In a further aspect, there is provided the use of AON1 (or composition comprising said oligonucleotide) as described in the previous sections for use as a medicament or part of therapy, or applications in which said AON1 exerts its activity intracellularly.

Preferably, AON1 or composition comprising AON1 is for use as a medicament or part of a therapy for preventing, delaying, curing, ameliorating and/or treating HD. All aspects and embodiments in relation to AON1 and a composition comprising it have already been defined herein.

### Method

In a further aspect, there is provided a method for treating, delaying, ameliorating and/or curing a condition or a disease as defined in the previous section in an individual, in a cell, tissue or organ of said individual. The method comprises intrathecally administering the AON1 as defined earlier herein at a dose as defined earlier herein. The disease or condition is Huntington.

In an embodiment of this method:
at least a single dose of 20 to 60 mg of AON1
preferably at least a single dose of 35 to 45 mg of AON1,
and more preferably at least a single dose of 40 mg of AON1 or more preferably at least a single dose of 60 mg of AON1
or a composition comprising said oligonucleotide is intrathecally administered to a human patient.

Each of the dose may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60.

In an embodiment of this method:
at least two doses of 20 to 40 mg of AON1,
preferably at least two doses of 35 to 45 mg of AON1,
more preferably at least two doses of 40 mg or more preferably at least two doses of 20 mg of AON1
or a composition comprising aid oligonucleotide is intrathecally administered to a human patient.

Each of the two doses may be 20, 21, 22, 23, 24, 25, 26, 27, 28, 29, 30, 31, 32, 33, 34, 35, 36, 37, 38, 39, 40, 41, 42, 43, 44, 45.

In an embodiment, of this method
at least three doses of 20 to 40 mg of AON1,
preferably at least three doses of 35 to 45 mg of AON1,
more preferably at leas three doses of 40 mg or more preferably at least three doses of 20 mg of AON1
or a composition comprising aid oligonucleotide is intrathecally administered to a human patient.

Each of the three doses may be 20, 21, 22, 23, 24, 25, 26, 27, 28, 29, 30, 31, 32, 33, 34, 35, 36, 37, 38, 39, 40, 41, 42, 43, 44, 45.

In an embodiment, AON1 is for use for treating, delaying, ameliorating and/or curing Huntington as follows: at least three doses of 20 to 40 mg, preferably at least three doses of 35 to 45 mg, more preferably at least three doses of 40 mg or more preferably at least three doses of 20 mg of this oligonucleotide are intrathecally and sequentially administered to a human patient.

In a preferred embodiment, the method for treating, curing, ameliorating and/or delaying a condition or disease as defined in the previous section in an individual, in a cell, tissue or organ of said individual comprises intrathecally administering at least a single dose of 35 to 45 mg of AON1 (preferably at least a 40 mg single dose of AON1) or a composition comprising AON1 to said individual or a subject in the need thereof as earlier disclosed herein.

The method according to the invention wherein AON1 or a composition comprising it may be suitable for administration to a cell, tissue and/or an organ in vivo of individuals affected by any of the herein defined diseases or at risk of developing said disease, and may be administered in vivo, ex vivo or in vitro. An individual or a subject in need is preferably a mammal, more preferably a human being.

Within the context of oligonucleotides for use, compositions for use, methods and uses according to the invention, an effective amount or therapeutically (and/or prophylactically) effective amount may be administered. Methods and uses of the invention will be later defined herein.

As used herein, an “effective amount” is an amount sufficient to exert beneficial or desired results. Accordingly, a “therapeutically effective amount” (prophylactically effective amount) is an amount that, when administered to a subject in need thereof, is sufficient to exert some therapeutic (prophylactic) effect as described herein, such as, but not limited to at least one of the below defined activities:
reducing or silencing or decreasing the translation rate of said mutant HTT transcript and thus the amount of the corresponding mutant HTT protein,
reducing or decreasing or lowering a mutant HTT protein level,
the level of Neurofilament light (Nf-L) is not increased (is preferably decreased) in blood and/or in the cerebrospinal fluid of the human patient.

An amount that is “therapeutically effective” (and/or prophylactically effective) will vary from subject to subject, depending on the age, the disease progression and overall general condition of the individual. An appropriate “therapeutically effective” (and/or prophylactically effective) amount in any individual case may be determined by the skilled person using routine experimentation, such as the methods described later herein.

The ranges of concentration or dose of oligonucleotide or composition as given above are preferred concentrations or doses for in vivo uses. The skilled person will understand that the dose of oligonucleotide used may further vary and may need to be optimised any further.

In this document and in its claims, the verb “to comprise” and its conjugations is used in its non-limiting sense to mean that items following the word are included, but items not specifically mentioned are not excluded. The verb “to comprise” is synonymous with the verb “to have” unless otherwise indicated. In addition the verb “to consist” may be replaced by “to consist essentially of” meaning that an oligonucleotide or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated. All patent and literature references cited in the present specification are hereby incorporated by reference in their entirety.

### General Information

Unless stated otherwise, all technical and scientific terms used herein have the same meaning as customarily and ordinarily understood by a person of ordinary skill in the art to which this invention belongs, and read in view of this disclosure.

### DEFINITIONS

Throughout the application, the words “binds”, “targets”, “hybridizes” could be used interchangeably when used in the context of an antisense oligonucleotide which is reverse complementary to a part of a pre-mRNA as identified herein. In the context of the invention, “hybridizes” or “binds” is used under physiological conditions in a cell, preferably a human cell unless otherwise indicated.

Throughout the application, the expressions “capable of”, “able to” could be used interchangeably when used in the context of an antisense oligonucleotide which is reverse complementary to a part of a pre-mRNA and is capable of hybridizing wich said pre-mRNA.

As used herein, “hybridization” refers to the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymine, 5-methyluracil and uracil which pair through the formation of hydrogen bonds. The natural base guanine is nucleobase complementary to the natural bases cytosine and 5-methyl-cytosine. Hybridization can occur under varying circumstances. In particular, hybridization of an oligonucleotide of the invention with a targeted pre-mRNA can occur under varying circumstances. Similarly, binding of an oligonucleotide of the invention to a targeted pre-mRNA can occur under varying circumstances. Preferably, said hybridization or said binding is assessed under physiological conditions in a cell, more preferably in a human cell. An oligonucleotide of the invention is preferably said to be able to bind to, or capable of binding to, or able to hybridize with, or capable of hybridizing with, when said binding or hybridization occurs under physiological conditions in a cell, preferably a human cell.

As used herein, “nucleotide” refers to a nucleoside further comprising a modified or unmodified phosphate linking group or a non-phosphate internucleoside linkage.

As used herein, “nucleotide analogue” or “nucleotide equivalent” refers to a nucleotide, which comprises at least one modification with respect to the nucleotides naturally occurring in RNA, such as A, C, G and U. Such a modification may be an internucleoside linkage modification and/or a sugar modification and/or a base modification.

As used herein, “monomer” refers to a precursor in the synthesis of an oligomeric or polymeric compound. Also the monomeric unit or residue within such an oligomeric or polymeric compound is encompassed in the term “monomer”. Thus, “monomer” and “nucleotide residue” may be used interchangeably throughout the description. Within the context of the present invention, a monomer is preferably a nucleotide.

As used herein, “nucleobase” refers to the heterocyclic base portion of a nucleoside. Nucleobases may be naturally occurring or may be modified and therefore include, but are not limited to adenine, cytosine, guanine, uracil, thymine and analogues thereof such as 5-methyl-cytosine. In certain embodiments, a nucleobase may comprise any atom or group of atoms capable of hydrogen bonding to a base of another nucleic acid.

As used herein, “Tₘ” means melting temperature which is the temperature at which the two strands of a duplex nucleic acid separate. Tₘ is often used as a measure of duplex stability or the binding affinity of an antisense compound toward a complementary RNA molecule.

As used herein, “2′-modified” or “2′-substituted” refers to a nucleoside comprising a pentose sugar comprising a substituent at the 2′ position other than H or OH. 2′-modified nucleosides include, but are not limited to, bicyclic nucleosides wherein the bridge connecting two carbon atoms of the sugar ring connects the 2′ carbon and another carbon of the sugar ring; and nucleosides with non-bridging 2′-substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), wherein each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-O-Me”, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, the term “adenine analogue” means a chemically-modified purine nucleobase that, when incorporated into an oligomer, is capable of forming a base pair with either a thymine or uracil of a complementary strand of RNA or DNA. Preferably, such base pair is a Watson-Crick base pair, but analogues and slight deviations thereof are also considered allowable within the context of the present invention.

As used herein, the term “uracil analogue” means a chemically-modified pyrimidine nucleobase that, when incorporated into an oligomer, is capable of forming a base pair with either a adenine of a complementary strand of RNA or DNA. Preferably, such base pair is a Watson-Crick base pair, but analogues and slight deviations thereof are also considered allowable within the context of the present invention.

As used herein, the term “thymine analogue” means a chemically-modified pyrimidine nucleobase that, when incorporated into an oligomer, is capable of forming a base pair with an adenine of a complementary strand of RNA or DNA. Preferably, such base pair is a Watson-Crick base pair, but analogues and slight deviations thereof are also considered allowable within the context of the present invention.

As used herein, the term “cytosine analogue” means a chemically-modified pyrimidine nucleobase that, when incorporated into an oligomer, is capable of forming a base pair with a guanine of a complementary strand of RNA or DNA. For example, cytosine analogue can be a 5-methylcytosine. Preferably, such base pair is a Watson-Crick base pair, but analogues and slight deviations thereof are also considered allowable within the context of the present invention.

As used herein, the term “guanine analogue” means a chemically-modified purine nucleobase that, when incorporated into an oligomer, is capable of forming a base pair with a cytosine of a complementary strand of RNA or DNA. Preferably, such base pair is a Watson-Crick base pair, but analogues and slight deviations thereof are also considered allowable within the context of the present invention.

As used herein, the term “guanosine” refers to a nucleoside or sugar-modified nucleoside comprising a guanine or guanine analog nucleobase.

As used herein, the term “uridine” refers to a nucleoside or sugar-modified nucleoside comprising a uracil or uracil analog nucleobase.

As used herein, the term “thymidine” refers to a nucleoside or sugar-modified nucleoside comprising a thymine or thymine analog nucleobase.

As used herein, the term “cytidine” refers to a nucleoside or sugar-modified nucleoside comprising a cytosine or cytosine analog nucleobase.

As used herein, the term “adenosine” refers to a nucleoside or sugar-modified nucleoside comprising an adenine or adenine analog nucleobase.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

As used herein, “oligomeric compound” refers to a polymeric structure comprising two or more sub-structures. In certain embodiments, an oligomeric compound is an oligonucleotide. In certain embodiments, an oligomeric compound is a single-stranded oligonucleotide. In certain embodiments, an oligomeric compound is a double-stranded duplex comprising two oligonucleotides. In certain embodiments, an oligomeric compound is a single-stranded or double-stranded oligonucleotide comprising one or more conjugate groups and/or terminal groups.

As used herein, “conjugate” refers to an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to the parent compound such as an oligomeric compound. In certain embodiments, conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In certain embodiments, conjugates are terminal groups. In certain embodiments, conjugates are attached to a 3′ or 5′ terminal nucleoside or to an internal nucleoside of an oligonucleotide.

As used herein, “conjugate linking group” refers to any atom or group of atoms used to attach a conjugate to an oligonucleotide or oligomeric compound. Linking groups or bifunctional linking moieties such as those known in the art are amenable to the present invention.

As used herein, “antisense compound” refers to an oligomeric compound, at least a portion of which is at least partially complementary to, or at least partially directed to, a target nucleic acid to which it hybridizes and modulates the activity, processing or expression of said target nucleic acid.

As used herein, “expression” refers to the process by which a gene ultimately results in a protein. Expression includes, but is not limited to, transcription, splicing, post-transcriptional modification, and translation.

As used herein, “antisense oligonucleotide” refers to an antisense compound that is an oligonucleotide.

As used herein, “antisense activity” refers to any detectable and/or measurable activity attributable to the hybridization of an anti sense compound to its target nucleic acid. In certain embodiments, such activity may be an increase or decrease in an amount of a nucleic acid or protein. In certain embodiments, such activity may be a change in the ratio of splice variants of a nucleic acid or protein. Detection and/or measuring of antisense activity may be direct or indirect. In certain embodiments, antisense activity is assessed by observing a phenotypic change in a cell or animal.

As used herein, “target nucleic acid” refers to any nucleic acid molecule the expression, amount, or activity of which is capable of being modulated by an antisense compound. In certain embodiments, the target nucleic acid is DNA or RNA. In certain embodiments, the target RNA is miRNA, mRNA, pre-mRNA, non-coding RNA, or natural antisense transcripts. For example, the target nucleic acid can be a cellular gene (or mRNA transcribed from the gene) whose expression is associated with a particular disorder or disease state,

As used herein, “target mRNA” refers to a pre-selected RNA molecule that encodes a protein.

As used herein, “targeting” or “targeted to” refers to the association of an antisense compound to a particular target nucleic acid molecule or a particular region of nucleotides within a target nucleic acid molecule. An antisense compound targets a target nucleic acid if it is sufficiently reverse complementary to the target nucleic acid to allow hybridization under physiological conditions. In this context “sufficiently reverse complementary” may be at least 90%, 91%, 92%, 93%, 94%, 95%, 96%, 97%, 98%, 99% or 100% reverse complementary with said targeted nucleic acid molecule.

As used herein, “target site” refers to a region of a target nucleic acid that is bound by an antisense compound. In certain embodiments, a target site is at least partially within the 3′ untranslated region of an RNA molecule. In certain embodiments, a target site is at least partially within the 5′ untranslated region of an RNA molecule. In certain embodiments, a target site is at least partially within the coding region of an RNA molecule. In certain embodiments, a target site is at least partially within an exon of an RNA molecule. In certain embodiments, a target site is at least partially within an intron of an RNA molecule. In certain embodiments, a target site is at least partially within a miRNA target site of an RNA molecule. In certain embodiments, a target site is at least partially within a repeat region of an RNA molecule.

As used herein, “target protein” refers to a protein, the expression of which is modulated by an antisense compound. In certain embodiments, a target protein is encoded by a target nucleic acid. In certain embodiments, expression of a target protein is otherwise iNf-Luenced by a target nucleic acid.

As used herein, “complementarity” in reference to nucleobases refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases refers to a pair of nucleobases that do not form hydrogen bonds with one another or otherwise support hybridization.

As used herein, “complementary” in reference to linked nucleosides, oligonucleotides, or nucleic acids, refers to the capacity of an oligomeric compound to hybridize to another oligomeric compound or nucleic acid through nucleobase complementarity. In certain embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% complementary to a target nucleic acid or reverse complementarity to a target nucleic acid.

As used herein, “modulation” refers to a perturbation of amount or quality of a function or activity when compared to the function or activity prior to modulation. For example, modulation includes the change, either an increase (stimulation or induction) or a decrease (inhibition or reduction) in gene expression. As a further example, modulation of expression can include perturbing splice site selection of pre-mRNA processing, resulting in a change in the amount of a particular splice-variant present compared to conditions that were not perturbed. As a further example, modulation includes perturbing translation of a protein.

As used herein, “motif” refers to a pattern of modifications in an oligomeric compound or a region thereof. Motifs may be defined by modifications at certain nucleosides and/or at certain linking groups of an oligomeric compound.

As used herein, “the same modifications” refer to modifications relative to naturally occurring molecules that are the same as one another, including absence of modifications. Thus, for example, two unmodified DNA nucleoside have “the same modification,” even though the DNA nucleoside is unmodified.

As used herein, “type of modification” in reference to a nucleoside or a nucleoside of a “type” refers to the modification of a nucleoside and includes modified and unmodified nucleosides.

Accordingly, unless otherwise indicated, a “nucleoside having a modification of a first type” may be an unmodified nucleoside.

As used herein, “pharmaceutically acceptable salts” refers to salts of active compounds that retain the desired biological activity of the active compound and do not impart undesired toxicological effects thereto.

As used herein, the term “independently” means that each occurrence of a repetitive variable within a claimed oligonucleotide is selected independent of one another. For example, each repetitive variable can be selected so that (i) each of the repetitive variables are the same, (ii) two or more are the same, or (iii) each of the repetitive variables can be different.

As used herein, a zero (0) in a range indicating number of a particular unit means that the unit may be absent. For example, an oligomeric compound comprising 0-2 regions of a particular motif means that the oligomeric compound may comprise one or two such regions having the particular motif, or the oligomeric compound may not have any regions having the particular motif. In instances where an internal portion of a molecule is absent, the portions flanking the absent portion are bound directly to one another. Likewise, the term “none” as used herein, indicates that a certain feature is not present.

As used herein, “analogue” or “derivative” means either a compound or moiety similar in structure but different in respect to elemental composition from the parent compound regardless of how the compound is made. For example, an analogue or derivative compound does not need to be made from the parent compound as a chemical starting material.

### General Definitions Related to Oligonucleotides and to AON1

An oligonucleotide is known in the art to be a polymeric molecule, such as a DNA and/or RNA molecule, typically a single-stranded DNA and/or RNA molecule, consisting of repeating monomers. The monomeric units are typically nucleotides (RNA nucleotides or DNA nucleotides) or modified nucleotides (“nucleotide analogs”). The most common naturally occurring nucleotides in RNA are adenosine monophosphate (A), cytidine monophosphate (C), guanosine monophosphate (G), and uridine monophosphate (U). These consist of a pentose sugar ribose, a 5′-linked phosphate group which is linked via a phosphate ester, and a 1′-linked base. A nucleotide without a phosphate group is known as a nucleoside. The most common naturally occurring nucleotides in DNA are deoxyadenosine monophosphate (A), deoxycytidine monophosphate (C), deoxyguanosine monophosphate (G), and deoxythymidine monophosphate (T). These consist of a pentose sugar 2′-deoxyribose, a 5′-linked phosphate group which is linked via a phosphate ester, and a 1′-linked base. The abbreviations A, C, G, T and U as used herein may be used to refer to a nucleobase (or base), a corresponding nucleoside, or a corresponding nucleotide. The term “nucleotide” as used herein includes both naturally occurring nucleotides as well as nucleotide analogs (described in more detail later herein), unless indicated otherwise. The term “oligonucleotide” as used herein encompasses salt forms of an oligonucleotide or that possess a ionizable group. An ionizable group may be a base or acid and may be charged or neutral. An ionizable group may be present as ion pair with an appropriate counterion that carries opposite charges. Non-limiting examples of cationic counterions include sodium, potassium, cesium, Tris, lithium, calcium, magnesium, trialkylammonium, triethylammonium and tetraalkylammonium. Non-limiting examples of anionic counterions are chloride, bromide, iodide, lactate, mesylate, besylate, triflate, acetate, trifluoroacetate, dichloroacetate, tartrate, lactate and citrate. Examples of counterions have been described (e.g. Kumar, Pharm. Technol., 2008, 3, 128). In some embodiments, an oligonucleotide as described herein is an oligonucleotide salt, for example a sodium salt.

In an embodiment, AON1 is chirally pure as described in WO2014/010250.

AON1 is a non-naturally occurring oligonucleotide. For example, it comprises at least one modified sugar, modified base, or modified linkage as described in more detail later herein. The presence of modifications, including linkage, sugar, and base modifications, may provide AON1 with attractive properties such as improved stability and resistance properties to degradation by exonucleases. Furthermore, the presence of modifications may improve safety, bio-distribution, stability, cellular uptake, intracellular trafficking, target binding affinity, duplex stability, and immunogenicity compared to an oligonucleotide consisting of non-modified DNA and/or non-modified RNA nucleotides.

In some embodiments, AON1 described herein is an isolated oligonucleotide. The term “isolated” refers to the separation of a compound from other components present during its production. “Isolated” is not meant to exclude artificial or synthetic mixtures with other compounds or materials, or the presence of impurities that do not substantially interfere with the fundamental activity, and that may be present, for example, due to incomplete purification, or the addition of stabilizers. In the context of oligonucleotides, the term “isolated” may refer to a molecule that is separated from sequences with which it is immediately contiguous in a naturally occurring sequence. For example, an “isolated” oligonucleotide may comprise a DNA molecule inserted into a vector, such as a plasmid or virus vector. In the context of oligonucleotides, the term “isolated” may also refer to synthetic oligonucleotides, i.e. chemically synthesized oligonucleotides.

Chemical synthesis of oligonucleotides is routine in the art and provides rapid and inexpensive access to custom-made oligonucleotides of a desired sequence and a desired chemistry. The most common method is solid-phase synthesis using phosphoramidite chemistry. Reference is made to “Synthesis of Therapeutic Oligonucleotides”. Eds: Satoshi Obika, Mitsuo Sekine. Springer; 1st ed. 2018, Singapore, incorporated herein by reference. Thus, in some embodiments, an oligonucleotide as described herein may be a synthetic oligonucleotide.

### Sugar of the Oligonucleotide AON1

The sugar connects the base and the phosphate, and is therefore often referred to as the scaffold of the nucleotide. A modification in the pentose sugar is therefore often referred to as a scaffold modification. A sugar modification may therefore be called a scaffold modification. For severe modifications, the original pentose sugar might be replaced in its entirety by another moiety that similarly connects the base and the phosphate. Preferred sugars and scaffolds and modified sugars and scaffolds including artificial sugars and scaffolds are described later herein.

AON1 as described above comprises modified sugars or scaffolds (including artifical sugars or scaffolds). AON1 describes herein comprises modified RNA nucleotides or RNA nucleotide analogs. All the sugars of AON1 as described herein are modified or artificial sugars and are 2′-O-modified RNA, more preferably 2′-O-alkyl or 2′-O-(substituted)alkyl, even more preferably 2′-O-methyl (2′-Ome).

All RNA nucleotides of AON1 have a modified ribosyl moiety as described above. Thus, the RNA nucleotides present in AON1 are preferably selected from the group consisting of: 2′-O-modified RNA, more preferably 2′-O-alkyl or 2′-O-(substituted)alkyl, even more preferably 2′-O-methyl (2′-Ome) or 2′-O-(2-methoxy)ethyl (2′-MOE), most preferably 2′-O-methyl (2′-Ome).

### Base of the Oligonucleotide AON1

A base, sometimes called a nucleobase, may be selected from one of the natural DNA or RNA nucleobases (adenine, cytosine, guanine, thymine, and uracil). A base may also be a natural base analog (“modified base”), including artificial bases. Cytosine, thymine, and uracil are pyrimidine bases, and are generally linked to the scaffold through their 1-nitrogen. Adenine and guanine are purine bases, and are generally linked to the scaffold through their 9-nitrogen. Preferred bases and modified bases including artificial bases are described later herein.

As described above, AON1 comprises modified nucleobases (including artifical bases).

The term “base modification” or “modified base” as identified herein refers to the modification of a naturally occurring base in RNA (i.e. pyrimidine or purine base) or to the de novo synthesis of a base. This de novo synthesized base could be qualified as “modified” by comparison to an existing base.

If such a base is a modified base or if a base analog is being used, said modified base or base analog should preferably keep the same base pair specificity as the base it replaces. “Base pairing” refers to the binding of two bases (or nucleobases) to each other by hydrogen bonds. Specifically, a nucleobase analog replacing cytosine is capable of base pairing with guanine, a nucleobase analog replacing guanine is capable of base pairing with cytosine, a nucleobase analog replacing adenine is capable of base pairing with uracil and a nucleobase analog replacing uracil is capable of base pairing with adenine.

All the bases of AON1 as described herein are modified or artificial bases.

A preferred modified base is 5-methylcytosine As described above, all cytosines of AON1 have been modified this way or replaced by 5-methylcytosine.

### Internucleoside Linkage of the Oligonucleotide AON1

A nucleotide is generally connected to neighbouring nucleotides through condensation of its 5′-phosphate moiety to the 3′-hydroxyl moiety of the neighbouring nucleotide monomer. Similarly, its 3′-hydroxyl moiety is generally connected to the 5′-phosphate of a neighbouring nucleotide monomer. This forms phosphodiester bonds. The phosphodiesters and the scaffold form an alternating copolymer. The bases are grafted to this copolymer, namely to the scaffold moieties. Because of this characteristic, the alternating copolymer formed by linked monomers of an oligonucleotide is often called the backbone of the oligonucleotide. Because the phosphodiester bonds connect neighbouring monomers together, they are often referred to as backbone linkages, internucleoside linkages or simply linkages or simply backbone. It is understood that when a phosphate group is modified so that it is instead an analogous moiety such as a phosphorothioate, such a moiety is still referred to as the “backbone linkage”, “internucleoside linkage”, or simply “linkage” or simply “backbone” of the monomer. This is referred to as a linkage modification. In general terms, the backbone of an oligonucleotide is thus comprised of alternating scaffolds and (backbone) linkages.

Thus an oligonucleotide having 10 nucleotides may contain 9 linkages, linking the 10 ribose units of the 10 nucleotides together. Additionally, there may be one or more last linkage(s) present at one or both sides of the oligonucleotide, which is only connected to one nucleotide. The terms “linkage”, “internucleoside linkage”, “backbone linkage” and “backbone” are also meant to indicate such a pendant linkage. In some embodiments, at least one of the linkages in the backbone of the oligonucleotide according to the invention is modified.

All internucleotide linkages of AON1 are modified or artificial internucleotide linkages, and are phosphorothioate internucleoside linkages. This means that all internucleoside linkages in AON1 are modified or artificial, and are phosphorothioate internucleoside linkages.

### Combination of Modifications in the Oligonucleotide AON1

It is customary to combine modified and/or artificial internucleoside linkages, nucleobases and sugar or scaffold moieties, such as those described above, in the same molecule.

AON1 consists of 2′-O-methyl RNA nucleotide residues, has a backbone wherein all phosphate moieties are replaced by phosphorothioate, and its cytosines have been replaced by 5-methyl cytosine.

AON1 is a modified RNA molecule. AON1 is a single stranded oligonucleotide.

In some embodiments, AON1 comprises or consists of SEQ ID NO:1. Also encompassed are oligonucleotides having a base sequence comprising or consisting of a sequence having up to 10, 9, 8, 7, 6, 5, 4, 3, 2 or 1 mutation(s) compared to the base sequence of SEQ ID NOs:1. Mutations include additions, insertions, deletions and substitutions. Also encompassed are oligonucleotides having a base sequence comprising or consisting of a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NOs: 1. Mutations include additions, insertions, deletions and substitutions. A preferred level of sequence identity is 80%. Another preferred level of sequence identity is 85%. Another preferred level of sequence identity is 90%. Another preferred level of sequence identity is 95%. Another preferred level of sequence identity is 97%.

It is understood that, in the context of any of the olignucleotide described throughout this invention, the term “comprising” may be replaced with the term “consisting essentially of” or “consisting”. In other words, in some embodiments, the oligonucleotide described herein consist essentially of a sequence that is capable of hybridizing with a region in the RNA of Huntingtin comprising said repetitive unit (CAG)n, or consist of a sequence that is capable of hybridizing with a region in said target RNA molecule comprising said repetitive unit.

### Length and Complementarity

AON1 comprises a sequence that is capable of hybridising with a region in a Huntingtin transcript comprising a repetitive nucleotide unit (CAG)n. “Hybridisation” as used herein typically refers to specific hybridisation, and excludes non-specific hybridisation. Thus, in some embodiments, AON1 comprises a sequence that is capable of specifically hybridising with a region in the Huntingtin RNA molecule comprising said repetitive nucleotide unit (CAG)n. Preferably, hybridisation is assessed under physiological conditions in a cell as described herein, more preferably in a human cell as described herein. Typically, a sequence that is capable of hybridising with a region in the Huntingtin RNA molecule comprising a repetitive nucleotide unit (CAG)n may be a sequence that has a certain level of complementarity with the Huntingtin RNA molecule. Perfect or full complementarity is not required, as long as the complementarity is sufficient to allow hybridisation, i.e. the formation of a double-stranded complex with the target RNA molecule. In some embodiments, oligonucleotides as described herein comprise a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% complementary with a sequence of the target RNA molecule. A preferred level of complementarity is at least 90%. Another preferred level of complementarity is at least 95%. A most preferred level of complementarity is 100%.

AON1 as described herein has a length of 21 nucleotides. In some embodiments, an oligonucleotide as described herein has a length of 16, 17, 18, 19, 20, 21, 22, 23, 24, 25, 26, 27, 28, 29, 30, 31, 32, 33, 34, 35, 36, 37, 38, 39, 40, 41 or 42 nucleotides, preferably 20, 21, 22, 23, 24, nucleotides

The following examples are offered for illustrative purposes only, and are not intended to limit the scope of the present invention in any way

### LEGENDS TO THE FIGURE

FIG. 1 Protocol overview of the schedule of assessment of the trial of the oligonucleotide AON1 with SEQ ID NO:1: A phase 1/2a, open-label clinical trial study to investigate the safety, tolerability and expected therapeutic efficacy of the intrathecally administered oligonucleotide AON1 in participants with early manifest Huntington disease (HD). Patients diagnosed with HD are treated four times in total with AON1, each time with a dosage of 40 mg on day 1, 29, 57 and 85 respectively. Cerebroventricular spinal fluid (CSF) samples are collected on day 1, 29, 57, 85, 120, 204, 253 and 337. MRI analysis is performed prior to dosing, at day 57, 120, 204, 253, 337 and 421. Dosing period is from day 1 to day 85, post dosing and safety follow-up period is from day 86 up to day 421.

FIG. 2 Baseline demographics of earlymanifest HD patients. The age, gender, CAG repeats, TMS, DCL and year of diagnosis are shown. CAG=cytosine-adenine-guanine; DCL=diagnostic confidence level; LLOQ=lower limit of quantification; TMS=total motor score.

FIG. 3 Baseline CSF mHTT (fM) levels in 6 early manifest HD patients. Baseline CSF mHTT levels (fM) of all the six HD patients, measured on day 1, are shown. Baseline CSF mHTT levels in two participants are below the lower limit of quantification (23 fM). CSF=cerebrospinal fluid; mHTT=mutant Huntingtin protein; fM=femtomolar.

FIG. 4 Mean CSF mHTT % change from baseline. Two participants with CSF mHTT (fM) levels values below LLOQ (23.9 fM) at baseline were excluded from time course analysis. % Change of CSF mHTT levels (fM) compared to baseline CSF mHTT levels (fM) are monitored in the other four participants. The data showed a lowering of CSF mHTT protein in HD participants treated with 40 mg of AON1 on day 1, 29, 57 and 85 respectively. CSF=cerebrospinal fluid; mHTT=mutant Huntingtin protein.

FIG. 5 Mean % change in Nf-L. Nf-L levels are measured on day 1 (n=6), 29(n=6), 57(n=5), and 85(n=4). Mean % changes in Nf-L on day 29, 57 and 85 compared to day 1 are calculated and shown. Nf-L: neurofilament light marker, a marker of neurodegeneration.

FIG. 6 Mean CSF mHTT/Total HTT % change from baseline.

CSF of the two participants that were excluded from the analysis in FIG. 4 was remeasured with the more sensitive Nucleic Acid Linked Immuno-Sandwich Assay (NULISA) (Ashton N.J. et al., 2025, Alzheimer's Dement. 21:e14621). Measurements of both mutant and total HTT protein levels were included, allowing a ratio of both biomarkers to be calculated. This is relevant given that AON1 has an allele preferential action and has a more profound effect on reduction of mutant HTT protein levels than on wild type HTT protein levels.

The data showed a lowering of the ratio of mutant/total HTT in CSF of these 2 HD participants treated with 40 mg of AON1 on day 1, 29, 57 and 85 respectively. CSF=cerebrospinal fluid; mHTT=mutant Huntingtin protein, total HTT=the sum of mutant and wild type Huntingtin protein.

### EXAMPLE

### Example 1

The oligonucleotide AON1 with SEQ ID NO:1 ((CUG)7 consisting of 2′-O-methyl phosphorothioate RNA wherein all of its cytosines have been replaced by 5-methylcytosine) has been tested in an open-label phase 1/2a trial.

### Trial Population

The trial population comprises generally ambulatory participants with early manifest HD. Patients have been classified using the TFC, the UHDRS and the CAG repeat length.

Early manifest HD: Stage I disease with a Total Functional Capacity (TFC) Score of ≥11 and ≤13 and a Unified Huntington's Disease Rating Scale (UHDRS) Diagnostic Confidence Level (DCL) of 4. c. Patients have good general health. FIG. 2 provides an overview of the demographics of the early manifest HD patients enrolled in this study.

Patients have ≥36 CAG repeats in HTT. FIG. 2 provides an overview of the demographics of the patients enrolled in this study.

HTT patients do not have one or more pathogenic mutation(s) in another polyQ disease gene, i.e., ATXN2, CACNA1A, ATXN7, TBP, AR, ATN1, ATX3, ATXN1 and ATXN3.

Pathogenic mutations are defined as: ≥41 contiguous, uninterrupted CAG repeats in ATXN1; ≥61 repeats in ATXN3; ≥38 CAG repeats in AR; ≥48 CAG repeats in ATN1; ≥33 CAG repeats in ATXN2; ≥34 CAG repeats in ATXN 7; ≥20 CAG repeats in CACNA1A; ≥41 CAG repeats in TBP.

The human pre-mRNA of these polyQ genes is identified as: HTT (SEQ ID NO: 2), ATXN1 (SEQ ID NO:3), ATXN2 (SEQ ID NO: 4) ATXN3 (SEQ ID NO: 5), CACNA1A (SEQ ID NO:6), ATXN7 (SEQ ID NO: 7), TBP (SEQ ID NO: 9), AR (SEQ ID NO: 10) or ATN1 (SEQ ID NO: 11) genes.

For all participants, the polyQ disease genes HTT, ATXN1, ATXN2, ATXN3, ATXN7, CACNA1A, TBP, AR, and ATN1 encoding for the nine known pathogenic polyQ proteins (both alleles) have been analysed by sequencing during the screening period.

This analysis is to confirm the presence of the disease-causing mutation in the gene affected in the individual participants, i.e. HTT in patients with HD, and the absence of any CAG repeat expansion mutation in any of the other polyQ disease genes.

### Experimental Procedures

The total duration of trial participation for each participant is up to approximately 43 weeks, consisting of a screening period of up to 6 weeks, a 14-week dosing period and a 23-week post-dosing period.

### Screening Period (Week −6 to Day −2):

Written informed consent for the trial will be obtained prior to the conduct of any trial-related procedures including screening procedures, and inclusion/exclusion criteria will be evaluated to determine patient eligibility for the trial.

### Dosing Period (Week 1 to Week 14):

The oligonucleotide was administered four times as a single IT (intrathecal) lumbar bolus injection at a plasma dose of 40 mg to six early manifest HD patients. The oligonucleotide was administered as a single IT lumbar bolus injection using a 24G atraumatic needle inserted into the L3/L4 space.

The oligonucleotide was administrated on days 1, 29, 57 and 85 in each of the four dosing blocks:

The dosing period consisted of four dosing blocks:
Dosing Block 1: Days −1 to 3;
Dosing Block 2: Days 28 to 31;
Dosing Block 3: Days 56 to 59;
Dosing Block 4: Days 84 to 87.

It means a total dose of 160 mg was administered to each patient.

The oligonucleotide is formulated in artificial cerebrospinal fluid (aCSF). ACSF contains 150 mM Na, 3.0 mM K, 1.4 mM Ca, 0.8 mM Mg, 1.0 mM P, 155 mM Cl. It is supplied as a stock solution in a 10 mL clear glass vial, at a strength of 10 mg/mL and with an extractable volume of 10 mL. The stock solution needs to be diluted prior to administration with the diluent (aCSF). The diluent is provided in a 25 mL clear glass vial.

The oligonucleotide was administered intrathecally four times on Day 1, Day 29, Day 57 and Day 85 within the planned dosing blocks, which include the day before dosing, the day of dosing and the day after dosing, and phone contact on the following day. During the dosing period, the pre-dose cerebrospinal fluid (CSF) sampling and intrathecal administration of the oligonucleotide will be performed through lumbar puncture (LP). Neuroimaging safety assessment by MRI will be performed prior to start dosing block 4.

### Biochemical Markers

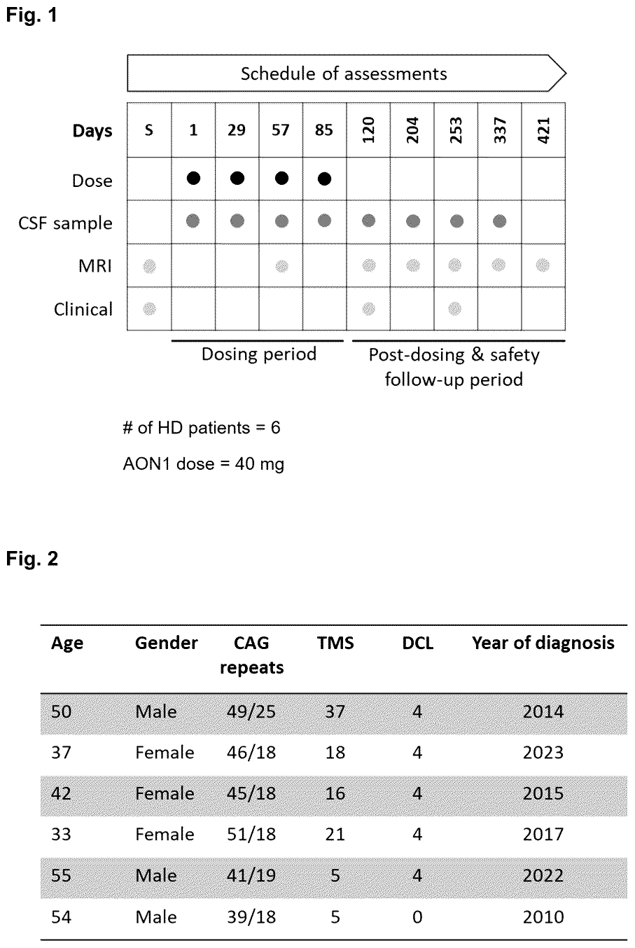

During the four dosing blocks, CSF and blood samples for safety and pharmacokinetics (PK) will be collected at specific time points. Cardiac monitoring using electrocardiograms (ECGs) and vital signs, including blood pressure, heart rate, respiratory rate, and temperature will be closely monitored after each IMP administration. Safety observation visits will occur 7 days after each dosing day. FIG. 1 provides an overview of the assessment of the patients.

CSF and blood samples have been collected to assess the following exploratory biochemical biomarkers:
Mutant HTT protein
Total ATXN1 protein, total ATXN3 protein and total HTT protein
Biomarkers of neurodegeneration: Nf-L, total tau, GFAP, UCH-L1
Biomarker of inflammation: C3a, IL-1β, IL-6, TNFα, YKL-40 (CH₃-L1)

Mutant HTT protein levels have been quantified in CSF using a highly sensitive and specific ELISA immunoassay and single molecule counting (SMC) with a femtomolar detection threshold, and a broad dynamic range: the ultrasensitive SMC method on the SMCxPRO platform using antibodies 2B7 (anti-HTT(7-13) mouse monoclonal antibody) and MW1 (anti-poly-Q mouse monoclonal antibody) (Wild E. et al., 2015, J Clin Invest, 125(5):1979-86).

Nf-L levels have been quantified in CSF using a digital immunoassay (Nf-L Simoa NF-light V2 Advantage Kit on the Quanterix SR-X platform).

The key exploratory, pharmacodynamic biomarker will be the change from baseline of the following mutant HTT protein in the CSF and in the blood.

The change from baseline in total ATXN1 protein, total ATXN3 protein and total HTT protein levels will be determined in CSF and the blood, if appropriate, as an indicator for potential off-target effects.

CSF and blood samples will be collected to assess markers of CNS cellular damage with neurofilament light chain (Nf-L) as the key exploratory biochemical biomarker. Additional markers that will be assessed for CNS cellular damage include total tau, GFAP and UCH-L1.

### Criteria for Evaluation:

### Primary Endpoints:

Incidence and dose relationships of treatment-related:
AEs (Adverse Events) are coded using the Medical Dictionary for Regulatory Activities (MedDRA) dictionary version 25.0 (March 2022),
Serious adverse events SAEs,
Adverse events of special interest (AESI),
Severe events (NCI-CTCAE Grade 3 or higher).
Changes in clinical safety parameters including physical and neurological examinations, vital signs, body weight, ECG, cardiac monitoring, suicidal ideation and behaviour risk monitoring by the Columbia Suicide Severity Rating Scale C-SSRS, and review of structural MRI scans
Changes in laboratory safety parameters in blood (haematology, haemostasis, clinical chemistry), CSF (cell counts, protein, glucose), and urine (urinalysis)
Adverse changes in clinical status based on exploratory clinical, biochemical and neuroimaging assessments.

### Exploratory Endpoints:

Changes in mutant HTT (in participants with HD) in CSF and blood.
Changes in biomarkers indicative of neurodegeneration, such as Nf-L, in CSF and blood.

### Results

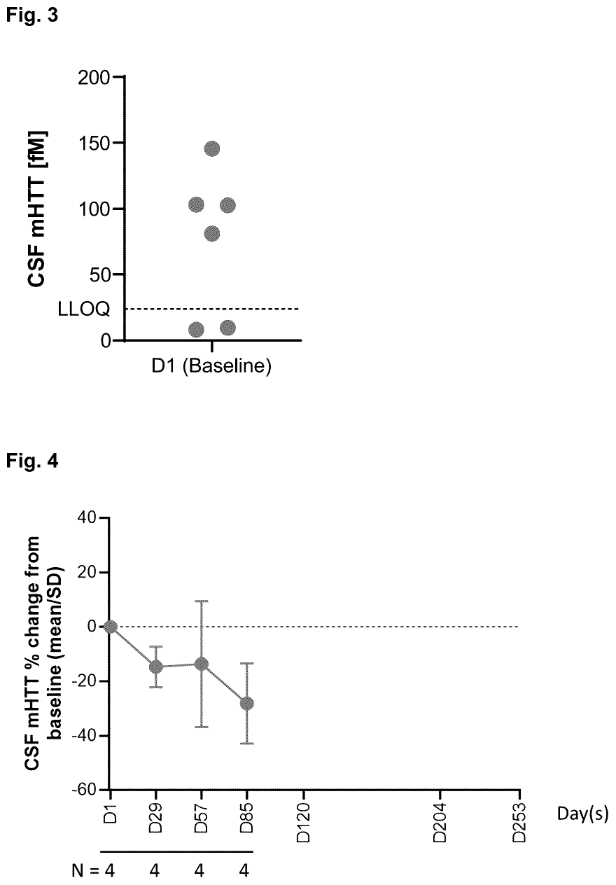

The level of mutant HTT protein in the six early manifest patients enrolled is provided in FIG. 3. Two of them had a level below detection, so their mutant HTT protein levels could not be assessed.

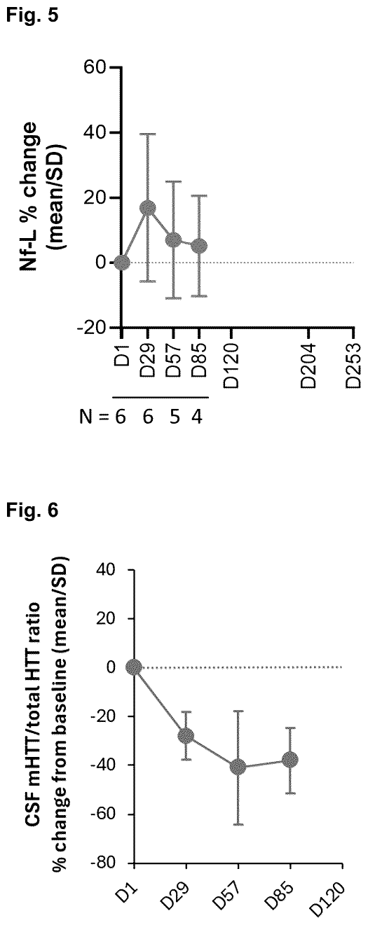

FIGS. 4 and 5 demonstrate that AON1 exerts a therapeutic effect in early manifest HD patients 28 days after only one single 40 mg dose: decrease of the mutant HTT protein level and stabilization of the Nf-L level have demonstrated. These effects are further enhanced 85 days after several 40 mg doses of AON1 have been administrated to the patients.

### Example 2

The oligonucleotide AON1 with SEQ ID NO:1 ((CUG)7 consisting of 2′-O-methyl phosphorothioate RNA wherein all of its cytosines have been replaced by 5-methylcytosine) has been tested in an additional open-label phase 1/2a trial.

All conditions used in example 1 also holds in example 2 unless otherwise indicated.

### Dosing Period (Week 1 to Week 26):

The oligonucleotide was administered one, two or three times as an IT (intrathecal) lumbar bolus injection at a plasma dose of 20 mg (three times), 40 mg (two times) and 60 mg (one time) to early manifest HD patients as indicated below. The oligonucleotide was administered as a single IT lumbar bolus injection using a 24G atraumatic needle inserted into the L3/L4 space.

### Dose-Level Cohort 1: 3 Times 20 mg of AON1 (Total Dose is 60 mg)

The oligonucleotide was administrated on days 1, 85 and 169 in each of the three dosing blocks:

The dosing period consisted of three dosing blocks:
Dosing Block 1: Days −1 to 3;
Dosing Block 2: Days 84 to 87;
Dosing Block 3: Days 168 to 171;

### Dose-Level Cohort 2: 2 Times 40 mg of AON1 (Total Dose is 80 mg)

The oligonucleotide was administrated on days 1 and 169 in each of the two dosing blocks: The dosing period consisted of two dosing blocks:
Dosing Block 1: Days −1 to 3;
Dosing Block 2: Days 168 to 171;

### Dose-Level Cohort 3: 1 Time 60 mg of AON1 (Total Dose is 60 mg)

The oligonucleotide was administrated on day 1 in each of the dosing block: The dosing period consisted of a single dosing blocks:
Dosing Block 1: Days −1 to 3;

The oligonucleotide is formulated in artificial cerebrospinal fluid (aCSF) as in example 1.

The oligonucleotide was administered intrathecally three, two or one time as indicated above within the planned dosing blocks, which include the day before dosing, the day of dosing and the day after dosing, and phone contact on the following day. On the day of dosing the pre-dose cerebrospinal fluid (CSF) sampling and intrathecal administration of the oligonucleotide will be performed through lumbar puncture (LP).

The total duration of trial participation for each participant in Dose-level Cohorts 1 (3 times 20 mg) is up to approximately 45 weeks, consisting of a screening period of up to 6 weeks, a 14-week dosing period, and a 25-week post-dosing period.

The total duration of trial participation for each participant in Dose-level Cohort 2 (2 times 40 mg) is up to approximately 58 weeks, consisting of a screening period of up to 7 weeks, a 26-week dosing period, and a 25-week post dosing period.

The total duration of trial participation for each participant in Dose-level Cohort 3 (one time 60 mg) is up to approximately 58 weeks, consisting of a screening period of up to 7 weeks, a single dosing followed by a 51-week period of non-dosing, observational visits (split into a 26-week ‘dosing period’ and a 25-week ‘post-dosing period’ for consistency in the SoA with Dose-level Cohort 2).

The Biochemical markers, the criteria for evaluation and the exploratory end-points are identical with the ones of example 1.

### Example 3

In FIG. 6, mutant HTT protein levels were quantified in CSF using an ultrasensitive and specific immunoassay with an attomolar detection threshold and a broad dynamic range: the Nucleic Acid Linked Immuno-Sandwich Assay (NULISA) (Ashton N.J. et al., 2025, Alzheimer's Dement. 21:e14621) on the ARGO HT system using antibodies 2B7 (anti-HTT(7-13) mouse monoclonal antibody) and MW1 (antipoly-Q mouse monoclonal antibody) for mutant HTT (Wild E. et al., 2015, J Clin Invest, 125(5):1979-86) and 2B7 (anti-HTT(7-13) mouse monoclonal antibody) and D7F7 (rabbit monoclonal antibody) for total HTT (Baldo B. et al., 2018, eNeuro 5(4) e0234-18.2018).

CSF of the two participants that were excluded from the analysis in FIG. 4 was remeasured with the more sensitive NULISA.

Measurements of both mutant and total HTT protein levels were included, allowing a ratio of both biomarkers to be calculated. This is relevant given that AON1 has an allele preferential action and has a more profound effect on reduction of mutant HTT protein levels than on wild type HTT protein levels.

The data showed a lowering of the ratio of mutant/total HTT in CSF of these 2 HD participants treated with 40 mg of AON1 on day 1, 29, 57 and 85 respectively. CSF=cerebrospinal fluid; mHTT=mutant Huntingtin protein, total HTT=the sum of mutant and wild type Huntingtin protein.

1-15. (canceled)

16. A method for treating, delaying, ameliorating and/or curing Huntington disease wherein at least a single dose of 20 to 60 mg of an oligonucleotide represented by SEQ ID NO:1 or a composition comprising said oligonucleotide is intrathecally administered to a human patient.

17. The method of claim 16, wherein at least a single dose of 35 to 45 mg of said oligonucleotide or of said composition is administered to the patient.

18. The method of claim 16, wherein at least two or at least three doses of 20 to 60 mg of said oligonucleotide or of said composition are administered to the patient.

19. The method of claim 16, wherein at least two or at least three doses of 35 to 45 mg of said oligonucleotide or of said composition are sequentially administered to the patient.

20. The method of claim 18, wherein the interval between each dose is ranged from 20 days to one year.

21. The method of claim 20, wherein the interval is 28, 84 or 168 days.

22. The method of claim 16, wherein the human patient is an early manifest Huntington disease patient.

23. The method of claim 16, wherein the human patient has at least 36 CAG repeats in the Huntington transcript.

24. The method of claim 23, wherein the number of CAG repeats is at least 37, 38, 39, 40, 41, 42, 43, 44, 45, 46, 47, 48, 49, 50, 51, 52, 53, or 54 CAG in said transcript.

25. The method of claim 16, wherein the human patient does not have a pathogenic mutation in another polyQ disease genes elected from the group consisting of ATXN2, CACNA1A, ATXN7, TBP, AR, ATN1, ATX3, ATXN1, PPP2R2B, and ATXN3, or a combination thereof.

26. The method of claim 16, wherein said oligonucleotide exhibits at least one of the below defined activities:
reducing or silencing or decreasing the translation rate of said mutant HTT transcript and thus the amount of corresponding mutant HTT protein,
reducing or decreasing or lowering a mutant HTT protein level.

27. The method of claim 16, wherein said oligonucleotide reduces the amount of said mutant Huntingtin protein in the cerebrospinal fluid of the human patient by at least 10% after:
at least one single 20 to 60 mg dose intrathecally administered,
at least two 20 to 60 mg doses intrathecally administered, and/or
at least three 20 to 60 mg doses intrathecally administered.

28. The method of claim 27, wherein said oligonucleotide reduces the amount of said mutant Huntingtin protein in the cerebrospinal fluid of the human patient by at least 10% after:
at least one single 35 to 45 mg dose intrathecally administered,
at least two 35 to 45 mg doses intrathecally administered, and/or
at least three 35 to 45 mg doses intrathecally administered.

29. The method of claim 16, wherein said oligonucleotide decreases or lowers the ratio of mutant/total HTT protein (or the ratio of mutant/wild type HTT protein) in the cerebrospinal fluid of a human patient by at least 5% after
at least one single 20 to 60 mg dose intrathecally administered,
at least two 20 to 60 mg doses intrathecally administered,
at least three 20 to 60 mg doses intrathecally administered, or
four 20 to 60 mg doses intrathecally administered.

30. The method of claim 29, wherein said oligonucleotide decreases or lowers the ratio of mutant/total HTT protein or the ratio of mutant/wild type HTT protein in the cerebrospinal fluid of the human patient by at least 5% after
at least one single 35 to 45 mg dose intrathecally administered,
at least two 35 to 45 mg doses intrathecally administered,
at least three 35 to 45 mg doses intrathecally administered, or
four 35 to 45 mg doses intrathecally administered.

31. The method of claim 16, wherein the level of Neurofilament light (Nf-L) is not increased in blood and/or in the cerebrospinal fluid of the human patient after at least one single 20 to 60 mg dose.

32. The method of claim 31, wherein the level of Neurofilament light (Nf-L) is not increased in blood and/or in the cerebrospinal fluid of the human patient after at least one single 35 to 45 mg dose.

33. The method of claim 31, wherein the level of Neurofilament light (Nf-L) is decreased in blood and/or in the cerebrospinal fluid of the human patient after at least two or three 20 to 60 mg doses.

34. The method of claim 33, wherein the level of Neurofilament light (Nf-L) is decreased in blood and/or in the cerebrospinal fluid of the human patient after at least two or three 35 to 45 mg doses.

35. The method of claim 16, wherein the composition comprises at least one excipient that may further aid in enhancing the targeting and/or delivery of said oligonucleotide to a tissue and/or cell of the human patient.

36. The method of claim 35, wherein the excipient is artificial cerebrospinal fluid.


SEQUENCE LISTING (11 sequences)

SEQ ID NO: 1 - RNA, 21 nt, synthetic construct
  source 1..21: mol_type = other RNA; note = antisense oligonucleotide (AON) (CUG)7
  modified_base 1: mod_base = m5c
  modified_base 4: mod_base = m5c
  modified_base 7: mod_base = m5c
  modified_base 10: mod_base = m5c
  modified_base 13: mod_base = m5c
  modified_base 16: mod_base = m5c
  modified_base 19: mod_base = m5c
  misc_feature 1..21: note = 2'-O-methyl phosphorothioate
ctgctgctgctgctgctgctg

SEQ ID NO: 2 - RNA, 13481 nt, Homo sapiens
  source 1..13481: mol_type = transcribed RNA; note = transcript of a HTT
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatca

SEQ ID NO: 3 - RNA, 10636 nt, Homo sapiens
  source 1..10636: mol_type = transcribed RNA; note = transcript of a ATXN1
gaggagagagcagagtataccgcagacatcatttctactacagtggcggagccgtacaggacctgtttcactgcagggggatccaaaacaagccccgtggagcagcagccagagcaacagcagccgcaagacattgtttctctccctctgcccccccttccccacgcaaccccagatccatttacactttacagttttacctcacaaaaactactacaagcaccaagctccctgatggaaaggagcatcgtgcatcaagtcaccagggtggtccattcaagctgcagatttgtttgtcatccttgtacagcaatctcctcctccactgccactacagggaagtgcatcacatgtcagcatactggagcatagtgaaagagtctattttgaagcttcaaacttagtgctgctgcagaccaggaacaagagagaaagagtggatttcagcctgcacggatggtcttgaaacacaaatggtttttggtctaggcgttttacactgagattctccactgccaccctttctactcaagcaaaatcttcgtgaaaagatctgctgcaaggaactgatagcttatggttctccattgtgatgaaagcacatggtacagttttccaaagaaattagaccattttcttcgtgagaaagaaatcgacgtgctgttttcatagggtatttctcacttctctgtgaaaggaagaaagaacacgcctgagcccaagagccctcaggagccctccagagcctgtgggaagtctccatggtgaagtataggctgaggctacctgtgaacagtacgcagtgaatgttcatccagagctgctgttggcggattgtacccacggggagatgattcctcatgaagagcctggatcccctacagaaatcaaatgtgactttccgtttatcagactaaaatcagagccatccagacagtgaaacagtcaccgtggaggggggacggcgaaaaatgaaatccaaccaagagcggagcaacgaatgcctgcctcccaagaagcgcgagatccccgccaccagccggtcctccgaggagaaggcccctaccctgcccagcgacaaccaccgggtggagggcacagcatggctcccgggcaaccctggtggccggggccacgggggcgggaggcatgggccggcagggacctcggtggagcttggtttacaacagggaataggtttacacaaagcattgtccacagggctggactactccccgcccagcgctcccaggtctgtccccgtggccaccacgctgcctgccgcgtacgccaccccgcagccagggaccccggtgtcccccgtgcagtacgctcacctgccgcacaccttccagttcattgggtcctcccaatacagtggaacctatgccagcttcatcccatcacagctgatccccccaaccgccaaccccgtcaccagtgcagtggcctcggccgcaggggccaccactccatcccagcgctcccagctggaggcctattccactctgctggccaacatgggcagtctgagccagacgccgggacacaaggctgagcagcagcagcagcagcagcagcagcagcagcagcagcatcagcatcagcagcagcagcagcagcagcagcagcagcagcagcagcagcacctcagcagggctccggggctcatcaccccggggtcccccccaccagcccagcagaaccagtacgtccacatttccagttctccgcagaacaccggccgcaccgcctctcctccggccatccccgtccacctccacccccaccagacgatgatcccacacacgctcaccctggggcccccctcccaggtcgtcatgcaatacgccgactccggcagccactttgtccctcgggaggccaccaagaaagctgagagcagccggctgcagcaggccatccaggccaaggaggtcctgaacggtgagatggagaagagccggcggtacggggccccgtcctcagccgacctgggcctgggcaaggcaggcggcaagtcggttcctcacccgtacgagtccaggcacgtggtggtccacccgagcccctcagactacagcagtcgtgatccttcgggggtccgggcctctgtgatggtcctgcccaacagcaacacgcccgcagctgacctggaggtgcaacaggccactcatcgtgaagcctccccttctaccctcaacgacaaaagtggcctgcatttagggaagcctggccaccggtcctacgcgctctcaccccacacggtcattcagaccacacacagtgcttcagagccactcccggtgggactgccagccacggccttctacgcagggactcaaccccctgtcatcggctacctgagcggccagcagcaagcaatcacctacgccggcagcctgccccagcacctggtgatccccggcacacagcccctgctcatcccggtcggcagcactgacatggaagcgtcgggggcagccccggccatagtcacgtcatccccccagtttgctgcagtgcctcacacgttcgtcaccaccgcccttcccaagagcgagaacttcaaccctgaggccctggtcacccaggccgcctacccagccatggtgcaggcccagatccacctgcctgtggtgcagtccgtggcctccccggcggcggctccccctacgctgcctccctacttcatgaaaggctccatcatccagttggccaacggggagctaaagaaggtggaagacttaaaaacagaagatttcatccagagtgcagagataagcaacgacctgaagatcgactccagcaccgtagagaggattgaagacagccatagcccgggcgtggccgtgatacagttcgccgtcggggagcaccgagcccaggtcagcgttgaagttttggtagagtatcctttttttgtgtttggacagggctggtcatcctgctgtccggagagaaccagccagctctttgatttgccgtgttccaaactctcagttggggatgtctgcatctcgcttaccctcaagaacctgaagaacggctctgttaaaaagggccagcccgtggatcccgccagcgtcctgctgaagcactcaaaggccgacggcctggcgggcagcagacacaggtatgccgagcaggaaaacggaatcaaccaggggagtgcccagatgctctctgagaatggcgaactgaagtttccagagaaaatgggattgcctgcagcgcccttcctcaccaaaatagaacccagcaagcccgcggcaacgaggaagaggaggtggtcggcgccagagagccgcaaactggagaagtcagaagacgaaccacctttgactcttcctaagccttctctaattcctcaggaggttaagatttgcattgaaggccggtctaatgtaggcaagtagaggcagcgtgggggaaaggaaacgtggctctcccttatcatttgtatccagattactgtactgtaggctaaaataacacagtatttacatgttatcttcttaattttaggtttctgttctaaccttgtcattagagttacagcaggtgtgtcgcaggagactggtgcatatgctttttccacgagtgtctgtcagtgagcgggcgggaggaagggcacagcaggagcggtcagggctccaggcatccccggggaagaaaggaacggggcttcacagtgcctgccttctctagcggcacagaagcagccgggggcgctgactcccgctagtgtcaggagaaaagtcccgtgggaagggtcctgcaggggtgcagggttgcacgcatgtgggggtgcacaggcgctgtggcggcgagtgagggtctctttttctctgcctccctctgcctcactctcttgctatcggcatgggccgggggggttcagagcagtgtcctcctggggttcccacgtgcaaaatcaacatcaggaacccagcttcagggcatcgcggagacgcgtcagatggcagatttggaaagttaaccatttaaaagaacatttttctctccaacatattttacaataaaagcaacttttaattgtatagatatatatttccccctatggggcctgactgcactgatatatattttttttaaagagcaactgccacatgcgggatttcatttctgctttttactagtgcagcgatgtcaccagggtgttgtggtggacagggaagcccctgctgtcatggccccacatggggtaaggggggttgggggtgggggagagggagagagcgaacacccacgctggtttctgtgcagtgttaggaaaaccaatcaggttattgcattgacttcactcccaagaggtagatgcaaactgcccttcagtgagagcaacagaagctcttcacgttgagtttgcgaaatctttttgtctttgaactctagtactgtttatagttcatgactatggacaactcgggtgccactttttttttttttcagattccagtgtgacatgaggaattagattttgaagatgagcatatattactatctttaagcatttaaaaatactgttcacactttattaccaagcatcttggtctctcattcaacaagtactgtatctcactttaaactctttggggaaaaaacaaaaacaaaaaaaactaagttgctttctttttttcaacactgtaactacatttcagctctgcagaattgctgaagagcaagatattgaaagtttcaatgtggtttaaagggatgaatgtgaattatgaactagtatgtgacaataaatgaccaccaagtactacctgacgggaggcacttttcactttgatgtctgagaatcagttcaaggcatatgcagagttggcagagaaactgagagaaaagggatggagaagagaatactcatttttgtccagtgtttttctttttaagatgaacttttaaagaaccttgcgatttgcacatattgagtttataacttgtgtgatattcctgcagtttttatccaataacattgtgggaaaggtttgggggactgaacgagcataaataaatgtagcaaaatttctttctaacctgcctaaactctaggccattttataaggttatgttcctttgaaaattcattttggtctttttaccacatctgtcacaaaaagccaggtcttagcgggctcttagaaactctgagaattttcttcagattcattgagagagttttccataaagacatttatatatgtgagcaagattttttttaaacaattactttattattgttgttattaatgttattttcagaatggcttttttttttctattcaaaatcaaatcgagatttaatgtttggtacaaacccagaaagggtatttcatagtttttaaacctttcattcccagagatccgaaatatcatttgtgggttttgaatgcatctttaaagtgctttaaaaaaaagttttataagtagggagaaatttttaaatattcttacttggatggctgcaactaaactgaacaaatacctgacttttcttttaccccattgaaaatagtactttcttcgtttcacaaattaaaaaaaaaatctggtatcaacccacattttggctgtctagtattcatttacatttagggttcaccaggactaatgatttttataaaccgttttctggggtgtaccaaaaacatttgaataggtttagaatagctagaatagttccttgactttcctcgaatttcattaccctctcagcatgcttgcagagagctgggtgggctcattcttgcagtcatactgcttatttagtgctgtattttttaaacgtttctgttcagagaacttgcttaatcttccatatattctgctcagggcacttgcaattattaggttttgtttttctttttgttttttagcctttgatggtaagaggaatacgggctgccacatagactttgttctcattaatatcactatttacaactcatgtggactcagaaaaacacacaccaccttttggcttacttcgagtattgaattgactggatccactaaaccaacactaagatgggaaaacacacatggtttggagcaataggaacatcatcataatttttgtggttctatttcaggtataggaattataaaataattggttctttctaaacacttgtcccatttcattctcttgcttttttagcatgtgcaatactttctgtgccaatagagtctgaccagtgtgctatatagttaaagctcattcccttttggctttttccttgtttggttgatcttccccattctggccagagcagggctggagggaaggagccaggagggagagagcctcccacctttcccctgctgcggatgctgagtgctggggcggggagccttcaggagccccgtgcgtctgccgccacgttgcagaaagagccagccaaggagacccgggggaggaaccgcagtgtcccctgtcaccacacggaatagtgaatgtggagtgtggagaggaaggaggcagattcatttctaagacgcactctggagccatgtagcctggagtcaacccattttccacggtcttttctgcaagtgggcaggcccctcctcggggtctgtgtccttgagacttggagccctgcctctgagcctggacgggaagtgtggcctgttgtgtgtgtgcgttctgagcgtgttggccagtggctgtggaggggaccacctgccacccacggtcaccactcccttgtggcagctttctcttcaaataggaagaacgcacagagggcaggagcctcctgtttgcagacgttggcgggccccgaggctcccagagcagcctctgtcaccgcttctgtgtagcaaacattaacgatgacaggggtagaaattcttcggtgccgttcagcttacaaggatcagccatgtgcctctgtactatgtccactttgcaatatttaccgacagccgtcttttgttctttctttcctgttttccatttttaaactagtaacagcaggccttttgcgtttacaatggaacacaatcaccaagaaattagtcagggcgaaaagaaaaaaataatactattaataagaaaccaacaaacaagaacctctctttctagggatttctaaatatataaaatgactgttccttagaatgtttaacttaagaattatttcagtttgtctgggccacactggggcagaggggggagggagggatacagagatggatgccacttacctcagatcttttaaagtggaaatccaaattgaattttcatttggactttcaggataattttctatgttggtcaacttttcgttttccctaactcacccagtttagtttgggatgatttgatttctgttgttgttgatcccatttctaacttggaattgtgagcctctatgttttctgttaggtgagtgtgttgggttttttccccccaccaggaagtggcagcatccctccttctcccctaaagggactctgcggaacctttcacacctctttctcagggacggggcaggtgtgtgtgtggtacactgacgtgtccagaagcagcactttgactgctctggagtagggttgtacaatttcaaggaatgtttggatttcctgcatcttgtggattactccttagataccgcatagattgcaatataatgctgcatgttcaagatgaacagtagctcctagtaatcataaaatccactctttgcacagtttgatctttactgaaatatgttgccaaaatttatttttgttgttgtagctctggattttgttttgttttgttttttaaggaaacgattgacaataccctttaacatctgtgactactaaggaaacctatttctttcatagagagaaaaatctccaatgcttttgaagacactaataccgtgctatttcagatatgggtgaggaagcagagctctcggtaccgaaggccgggcttcttgagctgtgttggttgtcatggctactgtttcatgaaccacaagcagctcaacagactggtctgttgccttctgaaaccctttgcacttcaatttgcaccaggtgaaaacagggccagcagactccatggcccaattcggtttcttcggtggtgatgtgaaaggagagaattacactttttttttttttaagtggcgtggaggcctttgcttccacatttgtttttaacccagaatttctgaaatagagaatttaagaacacatcaagtaataaatatacagagaatatacttttttataaagcacatgcatctgctattgtgttgggttggtttcctctcttttccacggacagtgttgtgtttctggcatagggaaactccaaacaacttgcacacctctactccggagctgagatttcttttacatagatgacctcgcttcaaatacgttaccttactgatgataggatcttttcttgtagcactataccttgtgggaatttttttttaaatgtacacctgatttgagaagctgaagaaaacaaaattttgaagcactcactttgaggagtacaggtaatgttttaaaaaattgcacaaaagaaaaatgaatgtcgaaatgattcattcagtgtttgaaagatatggctctgttgaaacaatgagtttcatactttgtttgtaaaaaaaaaaaagcagagaagggttgaaagttacatgtttttttgtatatagaaatttgtcatgtctaaatgatcagatttgtatggttatggcctggaagaattactacgtaaaaggctcttaaactatacctatgcttattgttatttttgttacatatagccctcgtctgagggaggggaactcggtattctgcgatttgagaatactgttcattcctatgctgaaagtacttctctgagctcccttcttagtctaaactcttaagccattgcaacttctttttcttcagagatgatgtttgacattttcagcacttcctgttcctataaacccaaagaatataatcttgaacacgaagtgtttgtaacaagggatccaggctaccaatcaaacaggactcattatggggacaaaaaaaaaaattatttcaccttctttccccccacacctcatttaaatggggggagtaaaaacatgatttcaatgtaaatgcctcattttattttagttttattttgatttttatttaatataaagaggccagaataaatacggagcatcttctcagaatagtattcctgtccaaaaatcaagccggacagtggaaactggacagctgtggggatattaagcacccccacttacaattcttaaattcagaatctcgtcccctcccttctcgttgaaggcaactgttctggtagctaactttctcctgtgtaatggcgggagggaacaccggcttcagtttttcatgtccccatgacttgcatacaaatggttcaactgtattaaaattaagtgcatttggccaataggtagtatctatacaataacaacaatctctaagaatttccataacttttcttatctgaaaggactcaagtcttccactgcagatacattggaggcttcacccacgttttctttccctttagtttgtttgctgtctggatggccaatgagcctgtctccttttctgtggccaatctgaaggccttcgttggaagtgttgtttacagtaatccttaccaagataacatactgtcctccagaataccaagtattaggtgacactagctcaagctgttgtcttcagagcagttaccaagaagctcggtgcacaggttttctctggttcttacaggaaccacctactctttcagttttctggcccaggagtggggtaaatcctttagttagtgcatttgaacttgatacctgtgcattcagttctgtgaatactgccctttttggcggggtttcctcatctccccagcctgaactgctcaactctaaacccaaattagtgtcagccgaaaggaggtttcaagatagtcctgtcagtatttgtggtgaccttcagattagacagtcttcatttccagccagtggagtcctggctccagagccatctctgagactcgtactactggatgttttaatatcagatcattacccaccatatgcctcccacaggccaagggaaaacagacaccagaacttgggttgagggcactaccagactgacatggccagtacagaggagaactagggaaggaatgatgttttgcaccttattgaaaagaaaattttaagtgcatacataatagttaagagcttttattgtgacaggagaacttttttccatatgcgtgcatactctctgtaattccagtgtaaaatattgtacttgcactagcttttttaaacaaatattaaaaaatggaagaattcatattctattttctaatcgtggtgtgtctatttgtaggatacactcgagtctgtttattgaattttatggtccctttctttgatggtgcttgcaggttttctaggtagaaattatttcattattataataaaacaatgtttgattcaaaatttgaacaaaattgttttaaataaattgtctgtataccagtacaagtttattgtttcagtatactcgtactaataaaataacagtgccaattgca

SEQ ID NO: 4 - RNA, 4652 nt, Homo sapiens
  source 1..4652: mol_type = transcribed RNA; note = transcript of a ATXN2
gccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 5 - RNA, 6923 nt, Homo sapiens
  source 1..6923: mol_type = transcribed RNA; note = transcript of a ATXN3
gagaggggcagggggcggagctggagggggtggttcggcgtgggggccgttggctccagacaaataaacatggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacagcagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgtcgttaagggtgatctgccagattgcgaagctgaccaactcctgcagatgattagggtccaacagatgcatcgaccaaaacttattggagaagaattagcacaactaaaagagcaaagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttcta

SEQ ID NO: 6 - RNA, 8646 nt, Homo sapiens
  source 1..8646: mol_type = transcribed RNA; note = transcript of CACNA1A
gatgtcccgagctgctatccccggctcggcccgggcagccgccttctgagcccccgacccgaggcgccgagccgccgccgcccgatgggctgggccgtggagcgtctccgcagtcgtagctccagccgccgcgctcccagccccggcagcctcagcatcagcggcggcggcggcggcggcggcgtcttccgcatcgttcgccgcagcgtaacccggagccctttgctctttgcagaatggcccgcttcggagacgagatgccggcccgctacgggggaggaggctccggggcagccgccggggtggtcgtgggcagcggaggcgggcgaggagccgggggcagccggcagggcgggcagcccggggcgcaaaggatgtacaagcagtcaatggcgcagagagcgcggaccatggcactctacaaccccatccccgtccgacagaactgcctcacggttaaccggtctctcttcctcttcagcgaagacaacgtggtgagaaaatacgccaaaaagatcaccgaatggcctccctttgaatatatgattttagccaccatcatagcgaattgcatcgtcctcgcactggagcagcatctgcctgatgatgacaagaccccgatgtctgaacggctggatgacacagaaccatacttcattggaattttttgtttcgaggctggaattaaaatcattgcccttgggtttgccttccacaaaggctcctacttgaggaatggctggaatgtcatggactttgtggtggtgctaacgggcatcttggcgacagttgggacggagtttgacctacggacgctgagggcagttcgagtgctgcggccgctcaagctggtgtctggaatcccaagtttacaagtcgtcctgaagtcgatcatgaaggcgatgatccctttgctgcagatcggcctcctcctattttttgcaatccttatttttgcaatcatagggttagaattttatatgggaaaatttcataccacctgctttgaagaggggacagatgacattcagggtgagtctccggctccatgtgggacagaagagcccgcccgcacctgccccaatgggaccaaatgtcagccctactgggaagggcccaacaacgggatcactcagttcgacaacatcctgtttgcagtgctgactgttttccagtgcataaccatggaagggtggactgatctcctctacaatagcaacgatgcctcagggaacacttggaactggttgtacttcatccccctcatcatcatcggctccttttttatgctgaaccttgtgctgggtgtgctgtcaggggagtttgccaaagaaagggaacgggtggagaaccggcgggcttttctgaagctgaggcggcaacaacagattgaacgtgagctcaatgggtacatggagtggatctcaaaagcagaagaggtgatcctcgccgaggatgaaactgacggggagcagaggcatccctttgatggagctctgcggagaaccaccataaagaaaagcaagacagatttgctcaaccccgaagaggctgaggatcagctggctgatatagcctctgtgggttctcccttcgcccgagccagcattaaaagtgccaagctggagaactcgaccttttttcacaaaaaggagaggaggatgcgtttctacatccgccgcatggtcaaaactcaggccttctactggactgtactcagtttggtagctctcaacacgctgtgtgttgctattgttcactacaaccagcccgagtggctctccgacttcctttactatgcagaattcattttcttaggactctttatgtccgaaatgtttataaaaatgtacgggcttgggacgcggccttacttccactcttccttcaactgctttgactgtggggttatcattgggagcatcttcgaggtcatctgggctgtcataaaacctggcacatcctttggaatcagcgtgttacgagccctcaggttattgcgtattttcaaagtcacaaagtactgggcatctctcagaaacctggtcgtctctctcctcaactccatgaagtccatcatcagcctgttgtttctccttttcctgttcattgtcgtcttcgcccttttgggaatgcaactcttcggcggccagtttaatttcgatgaagggactcctcccaccaacttcgatacttttccagcagcaataatgacggtgtttcagatcctgacgggcgaagactggaacgaggtcatgtacgacgggatcaagtctcaggggggcgtgcagggcggcatggtgttctccatctatttcattgtactgacgctctttgggaactacaccctcctgaatgtgttcttggccatcgctgtggacaatctggccaacgcccaggagctcaccaaggtggaggcggacgagcaagaggaagaagaagcagcgaaccagaaacttgccctacagaaagccaaggaggtggcagaagtgagtcctctgtccgcggccaacatgtctatagctgtgaaagagcaacagaagaatcaaaagccagccaagtccgtgtgggagcagcggaccagtgagatgcgaaagcagaacttgctggccagccgggaggccctgtataacgaaatggacccggacgagcgctggaaggctgcctacacgcggcacctgcggccagacatgaagacgcacttggaccggccgctggtggtggacccgcaggagaaccgcaacaacaacaccaacaagagccgggcggccgagcccaccgtggaccagcgcctcggccagcagcgcgccgaggacttcctcaggaaacaggcccgctaccacgatcgggcccgggaccccagcggctcggcgggcctggacgcacggaggccctgggcgggaagccaggaggccgagctgagccgggagggaccctacggccgcgagtcggaccaccacgcccgggagggcagcctggagcaacccgggttctgggagggcgaggccgagcgaggcaaggccggggacccccaccggaggcacgtgcaccggcaggggggcagcagggagagccgcagcgggtccccgcgcacgggcgcggacggggagcatcgacgtcatcgcgcgcaccgcaggcccggggaggagggtccggaggacaaggcggagcggagggcgcggcaccgcgagggcagccggccggcccggggcggcgagggcgagggcgagggccccgacgggggcgagcgcaggagaaggcaccggcatggcgctccagccacgtacgagggggacgcgcggagggaggacaaggagcggaggcatcggaggaggaaagagaaccagggctccggggtccctgtgtcgggccccaacctgtcaaccacccggccaatccagcaggacctgggccgccaagacccacccctggcagaggatattgacaacatgaagaacaacaagctggccaccgcggagtcggccgctccccacggcagccttggccacgccggcctgccccagagcccagccaagatgggaaacagcaccgaccccggccccatgctggccatccctgccatggccaccaacccccagaacgccgccagccgccggacgcccaacaacccggggaacccatccaatcccggcccccccaagacccccgagaatagccttatcgtcaccaaccccagcggcacccagaccaattcagctaagactgccaggaaacccgaccacaccacagtggacatccccccagcctgcccaccccccctcaaccacaccgtcgtacaagtgaacaaaaacgccaacccagacccactgccaaaaaaagaggaagagaagaaggaggaggaggaagacgaccgtggggaagacggccctaagccaatgcctccctatagctccatgttcatcctgtccacgaccaacccccttcgccgcctgtgccattacatcctgaacctgcgctactttgagatgtgcatcctcatggtcattgccatgagcagcatcgccctggccgccgaggaccctgtgcagcccaacgcacctcggaacaacgtgctgcgatactttgactacgtttttacaggcgtctttacctttgagatggtgatcaagatgattgacctggggctcgtcctgcatcagggtgcctacttccgtgacctctggaatattctcgacttcatagtggtcagtggggccctggtagcctttgccttcactggcaatagcaaaggaaaagacatcaacacgattaaatccctccgagtcctccgggtgctacgacctcttaaaaccatcaagcggctgccaaagctcaaggctgtgtttgactgtgtggtgaactcacttaaaaacgtcttcaacatcctcatcgtctacatgctattcatgttcatcttcgccgtggtggctgtgcagctcttcaaggggaaattcttccactgcactgacgagtccaaagagtttgagaaagattgtcgaggcaaatacctcctctacgagaagaatgaggtgaaggcgcgagaccgggagtggaagaagtatgaattccattacgacaatgtgctgtgggctctgctgaccctcttcaccgtgtccacgggagaaggctggccacaggtcctcaagcattcggtggacgccacctttgagaaccagggccccagccccgggtaccgcatggagatgtccattttctacgtcgtctactttgtggtgttccccttcttctttgtcaatatctttgtggccttgatcatcatcaccttccaggagcaaggggacaagatgatggaggaatacagcctggagaaaaatgagagggcctgcattgatttcgccatcagcgccaagccgctgacccgacacatgccgcagaacaagcagagcttccagtaccgcatgtggcagttcgtggtgtctccgcctttcgagtacacgatcatggccatgatcgccctcaacaccatcgtgcttatgatgaagttctatggggcttctgttgcttatgaaaatgccctgcgggtgttcaacatcgtcttcacctccctcttctctctggaatgtgtgctgaaagtcatggcttttgggattctgaattatttccgcgatgcctggaacatcttcgactttgtgactgttctgggcagcatcaccgatatcctcgtgactgagtttgggaatccgaataacttcatcaacctgagctttctccgcctcttccgagctgcccggctcatcaaacttctccgtcagggttacaccatccgcattcttctctggacctttgtgcagtccttcaaggccctgccttatgtctgtctgctgatcgccatgctcttcttcatctatgccatcattgggatgcaggtgtttggtaacattggcatcgacgtggaggacgaggacagtgatgaagatgagttccaaatcactgagcacaataacttccggaccttcttccaggccctcatgcttctcttccggagtgccaccggggaagcttggcacaacatcatgctttcctgcctcagcgggaaaccgtgtgataagaactctggcatcctgactcgagagtgtggcaatgaatttgcttatttttactttgtttccttcatcttcctctgctcgtttctgatgctgaatctctttgtcgccgtcatcatggacaactttgagtacctcacccgagactcctccatcctgggcccccaccacctggatgagtacgtgcgtgtctgggccgagtatgaccccgcagcttggggccgcatgccttacctggacatgtatcagatgctgagacacatgtctccgcccctgggtctggggaagaagtgtccggccagagtggcttacaagcggcttctgcggatggacctgcccgtcgcagatgacaacaccgtccacttcaattccaccctcatggctctgatccgcacagccctggacatcaagattgccaagggaggagccgacaaacagcagatggacgctgagctgcggaaggagatgatggcgatttggcccaatctgtcccagaagacgctagacctgctggtcacacctcacaagtccacggacctcaccgtggggaagatctacgcagccatgatgatcatggagtactaccggcagagcaaggccaagaagctgcaggccatgcgcgaggagcaggaccggacacccctcatgttccagcgcatggagcccccgtccccaacgcaggaagggggacctggccagaacgccctcccctccacccagctggacccaggaggagccctgatggctcacgaaagcggcctcaaggagagcccgtcctgggtgacccagcgtgcccaggagatgttccagaagacgggcacatggagtccggaacaaggcccccctaccgacatgcccaacagccagcctaactctcagtccgtggagatgcgagagatgggcagagatggctactccgacagcgagcactacctccccatggaaggccagggccgggctgcctccatgccccgcctccctgcagagaaccagaggagaaggggccggccacgtgggaataacctcagtaccatctcagacaccagccccatgaagcgttcagcctccgtgctgggccccaaggcccgacgcctggacgattactcgctggagcgggtcccgcccgaggagaaccagcggcaccaccagcggcgccgcgaccgcagccaccgcgcctctgagcgctccctgggccgctacaccgatgtggacacaggcttggggacagacctgagcatgaccacccaatccggggacctgccgtcgaaggagcgggaccaggagcggggccggcccaaggatcggaagcatcgacagcaccaccaccaccaccaccaccaccaccatcccccgccccccgacaaggaccgctatgcccaggaacggccggaccacggccgggcacgggctcgggaccagcgctggtcccgctcgcccagcgagggccgagagcacatggcgcaccggcagggcagtagttccgtaagtggaagcccagccccctcaacatctggtaccagcactccgcggcggggccgccgccagctcccccagaccccctccaccccccggccacacgtgtcctattcccctgtgatccgtaaggccggcggctcggggcccccgcagcagcagcagcagcagcagcagcagcagcagcagcaggcggtggccaggccgggccgggcggccaccagcggccctcggaggtacccaggccccacggccgagcctctggccggagatcggccgcccacggggggccacagcagcggccgctcgcccaggatggagaggcgggtcccaggcccggcccggagcgagtcccccagggcctgtcgacacggcggggcccggtggccggcatctggcccgcacgtgtccgaggggcccccgggtccccggcaccatggctactaccggggctccgactacgacgaggccgatggcccgggcagcgggggcggcgaggaggccatggccggggcctacgacgcgccaccccccgtacgacacgcgtcctcgggcgccaccgggcgctcgcccaggactccccgggcctcgggcccggcctgcgcctcgccttctcggcacggccggcgactccccaacggctactacccggcgcacggactggccaggccccgcgggccgggctccaggaagggcctgcacgaaccctacagcgagagtgacgatgattggtgctaagcccgggcgaggtggcgcccgcccggccccccacgcaccccacgcacacaccccacccgaggagccgcgcagaggccgcgggggcccagcacagagggcccgggagagggccagccgggagaccccagactctggagaggccagggctgggccacaagggtgtcccgcagagaccctcggccaaaagagaccctcctgggcagccacggcgccccccaaccagccccgatccccccacccacgacaggggctctcgggtgggaggcagggagcagacaaaccacacagccaagggatttgaattaactcagccatttttggagaactttggggaacatgaaaaaaaaaaaaaaaaaaaaaaaaaaaaacatttttaaaagaaaaaacggggagaaaaaaatagcttctattgatgagttttatcatctcaattgaatctttcctttccctgatgaagacagctggtggccgagtgcggcaaagaagccagaaggaaccagaatcccagtgccctacacccaccaccagacacactcacacccacacacgttctcagacacacacaagagtgcttgccggttataccaaaccctactattactgcctgcagaaatcaatttaaaaaaataataataacaataaacaattttaaaaaggacaaaaaaattaatgattgagaaaagaggcatttttttctgacatttggtcctgcttgaaacaacaaaagaagaagaaaaacccaccatcaccaccgattcctttgcttcttttttccttttttcctaccttgtttgaaaaccgtgggcttgggactgtgaattattgcatgacattcaaaaagaaaaaaaaaataaaaaaaagttgaatcaaa

SEQ ID NO: 7 - RNA, 7242 nt, Homo sapiens
  source 1..7242: mol_type = transcribed RNA; note = transcript of ATXN7
gaaaaagggtgaaagagaaacttggcgacctcccggaggagttcgcgaagcgaccaggagcgtgttgccatcgtcctcacccggcacccaattccaccacagagtcgggatttcgtcggtgatcgtgatggggtgcttttatttttctctttgattttcaaaaaatgtctatgtgactgtccctatcttaaggggaagttgaaagtgggggcgggggtgctcaatgagaaacgttgccttgtgtgtagttgtttggagcacactgcaaattatattggcatctctttccaaaagtcactttgattcaacttcgatagctttctcgtaaatggcacgtttaggtggtgagaggtggatgaggaaacaggcaccagtgcagctgatttgacctccagtgggatagatacgattagcaccaggatcgtgtctcattttgaacccagatctgaacagaattaagacgaacgagctttcacaattgcagcagatgaagatccattggtaaattgatcaggatttttggcctaccctccaaagaaaaggagcggaaagaatgtcggagcgggccgcggatgacgtcaggggggagccgcgccgcgcggcggcggcggcgggcggagcagcggccgcggccgcccggcagcagcagcagcagcagcagcagcagcagccgccgcctccgcagccccagcggcagcagcacccgccaccgccgccacggcgcacacggccggaggacggcgggcccggcgccgcctccacctcggccgccgcaatggcgacggtcggggagcgcaggcctctgcccagtcctgaagtgatgctgggacagtcgtggaatctgtgggttgaggcttccaaacttcctgggaaggacgggacagaattggacgaaagtttcaaggagtttgggaaaaaccgcgaagtcatggggctctgtcgggaagacatgccaatatttggtttctgtccagcccatgatgatttctacttggtggtgtgtaacgactgtaatcaggttgtcaaaccgcaggcatttcaatcacattatgaaagaagacatagctcatccagcaagccgcctttggccgttcctcccacttcagtattttccttcttcccttctctgtccaaaagcaaaggaggcagtgcaagtggaagcaaccgttcttccagtggaggtgttcttagcgcatcctcatcaagttccaagttgttgaaatcacccaaagagaaactgcagctcagggggaacaccaggccaatgcatcccattcagcaaagtagagttccccatggtagaatcatgacaccctctgtgaaagtggaaaagattcatccgaaaatggatggcacactactgaaatctgcggtggggccaacctgtcctgctactgtgagttccttagtcaagcctggccttaactgcccctcaataccaaagccaaccttgccttcacctggacagattctgaatggcaaagggcttcctgcaccgcccactctggaaaagaaacctgaagacaattccaataataggaaatttttaaataagagattatcagaaagagagtttgatcctgacatccactgtggggttattgatctcgacaccaagaagccctgcacccggtctttgacatgcaagacacattccttaacccagcgcagggctgtccagggtagaagaaaacgatttgatgtgttattagccgagcacaaaaacaaaaccagggaaaaggaattgattcgccatccggactctcagcaaccaccgcagcctctcagggacccgcatcccgcccctcctagaacgtcacaggagccgcaccaaaaccctcacggagtgattccttccgaatcaaagccttttgtagctagtaaacctaaacctcacacccccagtcttccaaggcctccaggctgccctgctcagcaaggtgggagtgcccccattgaccctcctccagtccatgaatctccacaccctcccctgcctgccactgagccagcttctcggttatccagtgaggagggcgaaggcgatgacaaagaagagtctgttgaaaaactggactgtcattattcaggtcatcatcctcagccagcatctttttgcacatttgggagccggcagataggaagaggctattacgtgtttgactccaggtggaatcgacttcgctgcgccctcaacctcatggtggagaagcatctgaatgcacagctatggaagaaaatcccaccagtgcccagtaccacctcacccatctccacacgtattcctcaccggacaaactctgtgccgacatcacaatgtggagtcagctatctggcagcagccaccgtctctacatccccagtcctgctctcatctacctgcatctccccaaatagcaaatcggtaccagctcatggaaccacactaaatgcacagcctgctgcttcaggggcgatggatcctgtgtgcagtatgcaatccagacaagtgtcctcttcatcctcatccccttccacgccctctggcctttcctcggttccttcctcccccatgtccaggaaacctcagaaattgaaatccagcaaatctttgaggcccaaggagtcttctggtaacagcactaactgtcaaaatgccagtagcagtaccagtggcggctcaggaaagaaacgcaaaaacagttccccactgttggttcactcttcctcctcctcttcctcctcctcctcttcttctcattccatggagtcttttaggaaaaactgtgtggctcactctgggcctccctacccctcaacggtaacatcttcccatagcatcggcctcaactgtgtgacgaataaagcaaatgcggtgaacgtccggcatgaccagtcagggaggggcccccccaccgggagccctgctgaatccatcaagaggatgagtgtgatggtgaacagcagtgattctactctttctcttgggccattcattcaccagtccaatgaactgcctgtcaactcccacggcagtttttcccactcacacactcctctagacaaactcataggaaagaaaagaaagtgctcacccagctcgagcagcatcaacaacagcagcagcaaacccacaaaggttgccaaagtgccagccgtgaacaatgtccacatgaaacacacaggcaccatcccaggggcacaaggactgatgaacagttccctccttcatcagccaaaggcacgtccctgacagctgaaaatagcacggggaggaataatgcggacacttttgaggacaagttacacctccactcagcactctggactccacgatgcctttgagtctgttttcccaacctcctgtgggcctcaagggtagaaacctgccgggctgttgttttaacgaggatttccctgaagctatgtctctagcagtgagtactcataaaggacactggatcaagttcagccaccgaattgcttttatcagtgttaaagtggtctgaactgcttgctaccaatctgtgagaagtttttgtttttgttttgttttttaacttgcagtatatcacagagccactcttcaagtagattggctgggcaaaagaatgttttggcaagagcgttactgtagacctttctccctccttccttttactaccatttttttttaacactgtcatctgtaggtcactctccagcagttaggcaccttaactggagaccagaaaccttccagagaacacagggctgcatcccgagcaaccctctgaagaagggaattaggctttagattttgatagcaatgttccaggaatgaaatatagatgttagcccaagacaccatgacaaaatagcccagccttttgagagtaatttgggaaaagaagctgtcagaagtttctaacttacaaactggtttgaaatttttgatgcccagacagcaagtataaatcattttggaggcttacttttcatgatacaaaagcaattctgtgtgatttttttttttaagaagaaagaaaatgcaagctagttttgagaaaggaaggccaaattgggtcgggggagggtgggagtgaggaagttaaaatcactatagggagaaaaaacttttttcaagatttccaaagagatgaaattttcttaatccttttaagttttcatagtaaacagtatggcagattgggttggttgtcctacctggtctatttttaaaagtcaccttttaaagtgacattattagatacacttaaatgtttccaaggcactctctacattacccttgtttttctctttggatactgtcctgggactaagtgtagatttctgcttcaagcacttctggcattgtgtgtttttgtatgcactccccttcatgccacttcagatgtttatttggatgtggttggggacgagagcagacaccaaggaaagggagttggagagaatgtaagtcctgacctgaaggtcttttgtgatgcatgtataggattgccctgacacacaccctcctttcttgggattataccagccatcttcctgagagttttggagccctctaggatattttttctagtaccccaccccccacccctaaagaaagaccttaatatgttaaaacagcattgcttggagaaggtgtcattgaattccgggacgagccggagcctttaaatgggtgcttccaccactacaggctcctgacacgagtaacaggcactgttgcttagaagaacacacgaagttgccgaacacagggtaaaatttccaaggcgctgatcgttgccctggccagggcctgatgagagccagtcagtacattctttttttcctacagttcttgggtttcaaacttcagtttcagggaatttcaagtcaacaacaggtagaatgaataaacttggttaccagcctaataatgtgaattgctacagaattattcttattatgtaagaaaacaaaaactttatgcagatactttagctataaattgatgtaaaatactgatttttttaaaggaaggagagaacagtatcttgttcaattattatgcaatcaatcagtaaatgtttttaaaatgatactacaggagagcttagtaaggagagggcatggatgggccagtttggcatagttgggagaaatcagtctggtttccatcccagtcggggaagagagaggtgagagggaatcagaacgtacctagttgattccttggtgacaagtgcaatggggtatgggtagaatttattttcagagccaagaggacttgatggttataaataaagttgcctttagcaatggaatttacagatcgatcatgttgttccgaaagatgtgaataggatccacaataacaagttgattcagactaatgtagatatttagattagcaagtattgaacatttgatttcttagactgaggttttaaatgaatttcattatttctcccggtaatacacagagcatcgggattaggaaattagccattttggattctgtctgccacaaacagacctattctaaacagtgctattaaattttagactgttgttcaaatattttattttctcctaaactactttttatggtgatgaataattaagaccatttaaaacacgggagtacaagtatttttttgaattaaattaacttgcaaaaaccaaatttgcagtggttgggttgtttctagacagactttggtatcaatttaaaaccaagataatttttatattctttccaatagaatttcatgacaactcctgcagttttcttaacctacaaaaaaaaaaaaaaagtgctgcaatgatgaacaaagaattatacaaaacctacttttgtatctttattttggaatttcttgttctattataaatatggaagtatccctatttcagaagacatattttgttaaaaatgaattgtacatatttaaatatgtatttttgcacaggtctttttttctgatatcctgttttggtacaattgagatcatctagtatttatttattaattaataaaagtaaatacctttttataatttgaagtggttcactgccaagccaatagttctagaacctgcctcctttacagttttaagggatgcctctgttctgtgaaaagtctttgtcccttttagtgtctcgggagtggattcatacagatgaagtgggcattgcttcttcctggttgcctggtttcccgatagactacatgtaactaagtgacacactgcttttcttttgttagtattttatgtgtgggagtatgtgactgcgtgtgtgtgtgcctgtgcgtgtgtgtgtatactcagcacatgtatgttactatgtgatgtggtttaaaactaatggaaaaaactgaaagtgcctgaaactagttttaagcttagacagaatctctttaaaaagacttgaatgttcagttgaacttttggagtttgctttttccacctaaatattgtttctaaaattttaggggcggagttgaaaaccaccaatgctggtaattttataaggtattttaaaattaagacctcttggttcatagtaattcaattggtgatggattgcctggtggcaccaagggttacagatctttttgtcagccagcaacttacacgatgtgtccgttttgttattcactcatgaaatacaatttaaaatatttaaaatattttgtattccaaaaatataatacaaagaagtacctctgagaacattgaaaaaaatgtataatttgaaaaatgtaacttataagggcagctgtcttcctgcaagagttctgttgccaaggaatttcttaatgtcgctcattctgtccagggggtttggggcgattgggctttgaaaaaatattcttaaaagtttgtttagcagaaaataatcacttttacattttgtgcaaaacattttacattttcagataatttaaatgagcactacatactgtcagtgtgaatgtagggccttgaaagcattttgcttttttttttttttttttttttttgagcttggttataaaagcaatctcctgtgttaaaaacgtgtgaatagtttgataatttgtacacataatcaagagcatctcagctggactttgtgttggctgctgtatagaacatgaacaaatgtcaagggatagaaaattactttggatatttaaaagagaagaaatatatagtctatttgttttgtaacaagtttctgtaaataaaataatttatactatttataagaaaaaaaaaaaaaaaaaaa

SEQ ID NO: 8 - RNA, 2300 nt, Homo sapiens
  source 1..2300: mol_type = transcribed RNA; note = transcript of PPP2R2B
ggccaggcaagcctgaatcctgtccctgccatctcgccactgcagctcgggtccagaaaggcaccattttgtcgcggctgcccgctctcccagggggaggagggatcttttttgcattttggagcggctgccaaggaggggaacctgttgggcatctccccagacccgcttgtgagcgcctccggggcgggcgggcgggaccagacccctcggggcacggcgtatcttggcacccggaggcagcggaggcaggcgcagcatcctcgctgggaactggagctggagtgagcgcaccgcgcgggaggagccgccgcagcctcgcagaacccgagtggaggaggtgacagctccattgccgggtttttattttttttctctccgcctccccgtctcctcctcaggctcggaccatggtgcagtcccactggctcccctgcccccctctcctgtgagactggctgcggggagggatcatggatacttgtctgccggcttctggttcccacgcaagtaagcctgctgtcaatggaggaggacattgatacccgcaaaatcaacaacagtttcctgcgcgaccacagctatgcgaccgaagctgacattatctctacggtagaattcaaccacacgggagaattactagcgacaggggacaaggggggtcgggttgtaatatttcaacgagagcaggagagtaaaaatcaggttcatcgtaggggtgaatacaatgtttacagcacattccagagccatgaacccgagttcgattacctgaagagtttagaaatagaagaaaaaatcaataaaataagatggctcccccagcagaatgcagcttactttcttctgtctactaatgataaaactgtgaagctgtggaaagtcagcgagcgtgataagaggccagaaggctacaatctgaaagatgaggagggccggctccgggatcctgccaccatcacaaccctgcgggtgcctgtcctgagacccatggacctgatggtggaggccaccccacgaagagtatttgccaacgcacacacatatcacatcaactccatatctgtcaacagcgactatgaaacctacatgtccgctgatgacctgaggattaacctatggaactttgaaataaccaatcaaagttttaatattgtggacattaagccagccaacatggaggagctcacggaggtgatcacagcagccgagttccacccccatcattgcaacaccttcgtgtacagcagcagcaaagggacaatccggctgtgtgacatgcgggcatctgccctgtgtgacaggcacaccaaattttttgaagagccggaagatccaagcaacagatcatttttctctgaaattatctcttcgatttcggatgtgaagttcagccacagtgggaggtatatcatgaccagggactacttgaccgtcaaagtctgggatctcaacatggaaaaccgccccatcgagacttaccaggttcatgactacctccgcagcaagctgtgttccctctatgaaaatgactgcatttttgataaatttgagtgtgtgtggaatgggtcagacagtgtcatcatgacaggctcctacaacaacttcttcaggatgttcgacagaaacaccaagcgtgatgtgacccttgaggcttcgagggaaaacagcaagccccgggctatcctcaaaccccgaaaagtgtgtgtggggggcaagcggagaaaagacgagatcagtgtcgacagtctggactttagcaaaaagatcttgcatacagcttggcatccttcagaaaatattatagcagtggcggctacaaataacctatatatattccaggacaaggttaactaggtggacaagttattacttaataatctcacatactgaatactagtcaaacaagtttttaaatgtttctttgggtcttcatttgatgcattgactttaatttccctatacaggaaatgattggaatagaattaaaaggagtccaacattcccagctccccagttctaagaaacttttgtcaaacccaataggtttgggacacttctgtttagaattgaaagctgccagctaacagtaattcttccatagttgacttgaacttctgatgcttttattgcccagttttctctggtgggtccagtgttttgttcctaggtgtctgctgcgataaaatgaggttgtctgtagtatttaaggagaaaagagataagttttttttaattaagcaattccatttgattgaaaaaaatcaacaaaaaataaacaccgtttactcttagacaaa

SEQ ID NO: 9 - RNA, 1921 nt, Homo sapiens
  source 1..1921: mol_type = transcribed RNA; note = transcript of TBP
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 10 - RNA, 10661 nt, Homo sapiens
  source 1..10661: mol_type = transcribed RNA; note = transcript of AR
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa

SEQ ID NO: 11 - RNA, 4367 nt, Homo sapiens
  source 1..4367: mol_type = transcribed RNA; note = transcript of ATN1
gacgccatactggacgccaagtgggaggaacttcaaggctgtcccctgcgggcctcccgctctgcttctgcgaaggtttcattgaaaacagatcctgcaaaagttccaggtgcccacactggaaacttggagatcctgcttcccagaccacagctgtggggaacttggggtggagcagagaagtttctgtattcagctgcccaggcagaggagaatggggtctccacagcctgaagaatgaagacacgacagaataaagactcgatgtcaatgaggagtggacggaagaaagaggcccctgggccccgggaagaactgagatcgaggggccgggcctcccctggaggggtcagcacgtccagcagtgatggcaaagctgagaagtccaggcagacagccaagaaggcccgagtagaggaagcctccaccccaaaggtcaacaagcagggtcggagtgaggagatctcagagagtgaaagtgaggagaccaatgcaccaaaaaagaccaaaactgagcaggaactccctcggccacagtctccctccgatctggatagcttggacgggcggagccttaatgatgatggcagcagcgaccctagggatatcgaccaggacaaccgaagcacgtcccccagtatctacagccctggaagtgtggagaatgactctgactcatcttctggcctgtcccagggcccagcccgcccctaccacccacctccactctttcctccttcccctcaaccgccagacagcacccctcgacagccagaggctagctttgaaccccatccttctgtgacacccactggatatcatgctcccatggagccccccacatctcgaatgttccaggctcctcctggggcccctccccctcacccacagctctatcctgggggcactggtggagttttgtctggacccccaatgggtcccaaggggggaggggctgcctcatcagtggggggccctaatgggggtaagcagcaccccccacccactactcccatttcagtatcaagctctggggctagtggtgctcccccaacaaagccgcctaccactccagtgggtggtgggaacctaccttctgctccaccaccagccaacttcccccatgtgacaccgaacctgcctcccccacctgccctgagacccctcaacaatgcatcagcctctccccctggcctgggggcccaaccactacctggtcatctgccctctccccacgccatgggacagggtatgggtggacttcctcctggcccagagaagggcccaactctggctccttcaccccactctctgcctcctgcttcctcttctgctccagcgccccccatgaggtttccttattcatcctctagtagtagctctgcagcagcctcctcttccagttcttcctcctcttcctctgcctcccccttcccagcttcccaggcattgcccagctacccccactctttccctcccccaacaagcctctctgtctccaatcagccccccaagtatactcagccttctctcccatcccaggctgtgtggagccagggtcccccaccacctcctccctatggccgcctcttagccaacagcaatgcccatccaggccccttccctccctctactggggcccagtccaccgcccacccaccagtctcaacacatcaccatcaccaccagcaacagcaacagcagcagcagcagcagcagcagcagcagcagcagcagcagcagcatcacggaaactctgggccccctcctcctggagcatttccccacccactggagggcggtagctcccaccacgcacacccttacgccatgtctccctccctggggtctctgaggccctacccaccagggccagcacacctgcccccacctcacagccaggtgtcctacagccaagcaggccccaatggccctccagtctcttcctcttccaactcttcctcttccacttctcaagggtcctacccatgttcacacccctccccttcccagggccctcaaggggcgccctaccctttcccaccggtgcctacggtcaccacctcttcggctaccctttccacggtcattgccaccgtggcttcctcgccagcaggctacaaaacggcctccccacctgggcccccaccgtacggaaagagagccccgtccccgggggcctacaagacagccaccccacccggatacaaacccgggtcgcctccctccttccgaacggggaccccaccgggctatcgaggaacctcgccacctgcaggcccagggaccttcaagccgggctcgcccaccgtgggacctgggcccctgccacctgcggggccctcaggcctgccatcgctgccaccaccacctgcggcccctgcctcagggccgcccctgagcgccacgcagatcaaacaggagccggctgaggagtatgagacccccgagagcccggtgcccccagcccgcagcccctcgccccctcccaaggtggtagatgtacccagccatgccagtcagtctgccaggttcaacaaacacctggatcgcggcttcaactcgtgcgcgcgcagcgacctgtacttcgtgccactggagggctccaagctggccaagaagcgggccgacctggtggagaaggtgcggcgcgaggccgagcagcgcgcgcgcgaagaaaaggagcgcgagcgcgagcgggaacgcgagaaagagcgcgagcgcgagaaggagcgcgagcttgaacgcagcgtgaagttggctcaggagggccgtgctccggtggaatgcccatctctgggcccagtgccccatcgccctccatttgaaccgggcagtgcggtggctacagtgcccccctacctgggtcctgacactccagccttgcgcactctcagtgaatatgcccggcctcatgtcatgtctcctggcaatcgcaaccatccattctacgtgcccctgggggcagtggacccggggctcctgggttacaatgtcccggccctgtacagcagtgatccagctgcccgggagagggaacgggaagcccgtgaacgagacctccgtgaccgcctcaagcctggctttgaggtgaagcctagtgagctggaacccctacatggggtccctgggccgggcttggatccctttccccgacatgggggcctggctctgcagcctggcccacctggcctgcaccctttcccctttcatccgagcctggggcccctggagcgagaacgtctagcgctggcagctgggccagccctgcggcctgacatgtcctatgctgagcggctggcagctgagaggcagcacgcagaaagggtggcggccctgggcaatgacccactggcccggctgcagatgctcaatgtgactccccatcaccaccagcactcccacatccactcgcacctgcacctgcaccagcaagatgctatccatgcagcctctgcctcggtgcaccctctcattgaccccctggcctcagggtctcaccttacccggatcccctacccagctggaactctccctaaccccctgcttcctcaccctctgcacgagaacgaagttcttcgtcaccagctctttgctgccccttaccgggacctgccggcctccctttctgccccgatgtcagcagctcatcagctgcaggccatgcacgcacagtcagctgagctgcagcgcttggcgctggaacagcagcagtggctgcatgcccatcacccgctgcacagtgtgccgctgcctgcccaggaggactactacagtcacctgaagaaggaaagcgacaagccactgtagaacctgcgatcaagagagcaccatggctcctacattggaccttggagcacccccaccctccccccaccgtgcccttggcctgccacccagagccaagagggtgctgctcagttgcagggcctccgcagctggacagagagtgggggagggagggacagacagaaggccaaggcccgatgtggtgtgcagaggtggggaggtggcgaggatggggacagaaagcgcacagaatcttggaccaggtctctcttccttgtcccccctgcttttctcctcccccatgcccaacccctgtggccgccgcccctcccctgccccgttggtgtgattatttcatctgttagatgtggctgttttgcgtagcatcgtgtgccacccctgcccctccccgatccctgtgtgcgcgccccctctgcaatgtatgccccttgccccttccccacactaataatttatatatataaatatctatatgacgctcttaaaaaaacatcccaaccaaaaccaaccaaacaaaaacatcctcacaactccccaggaaaaaaaaaaaaaaaaaa